(19) United States
(12) Patent Application Publication
(10) Pub. No.: US 2026/0224225 A1
(43) Pub. Date: Aug. 6, 2026

(54) EPICARDIAL CLIP ASSEMBLY

(71) Applicant: LSI Solutions, Inc., Victor, NY (US)

(72) Inventors: Jude S. Sauer, Pittsford, NY (US); Benjamin James Boseck, Rochester, NY (US); Matthew Wrona, Fairport, NY (US); Michael W Fitzsimmons, Rochester, NY (US)

(21) Appl. No.: 19/635,595

(22) Filed: Mar. 31, 2026

Related U.S. Application Data
(63) Continuation-in-part of application No. 18/612,814, filed on Mar. 21, 2024.
(60) Provisional application No. 63/453,976, filed on Mar. 22, 2023.
(60) Provisional application No. 63/467,747, filed on May 19, 2023.
(60) Provisional application No. 63/527,438, filed on Jul. 18, 2023.
(60) Provisional application No. 63/624,988, filed on Jan. 25, 2024.
(60) Provisional application No. 63/781,405, filed on Apr. 1, 2025.
(60) Provisional application No. 63/946,307, filed on Dec. 22, 2025.

(51) Int. Cl. A61B 17/122; A61B 17/00; A61B 17/128

(52) U.S. Cl. CPC A61B 17/122; A61B 17/1285; A61B 2017/00004

(57) ABSTRACT

An absorbable epicardial clip assembly for occluding a left atrial appendage includes a clip body assembly having an elongated first arm body, an elongated second arm body, and a hinge portion coupled the first and second arm bodies that is configured to pivot from a disengaged position to an engaged position, in which engagement surfaces are configured to sufficiently compress opposing tissue surfaces of a portion of the left atrial appendage to induce ischemic necrosis along the portion of the left atrial appendage and to cut off the blood flow distal to the portion of the left atrial appendage, thereby leading to left atrial appendage tissue death and reabsorption. The clip body assembly is made from a first absorbable material such that the clip body assembly is configured to degrade within a first period of time after being secured in the engaged position.

### CROSS-REFERENCE TO RELATED APPLICATIONS

This application is a continuation-in-part of U.S. patent application Ser. No. 18/612,814, filed Mar. 21, 2024, which claims the benefit of each of U.S. Provisional Patent Application No. 63/453,976, filed Mar. 22, 2023, U.S. Provisional Patent Application No. 63/467,747, filed May 19, 2023, U.S. Provisional Patent Application No. 63/527,438, filed Jul. 18, 2023, and U.S. Provisional Patent Application No. 63/624,988, filed Jan. 25, 2024, the contents of each which is hereby incorporated by reference in its entirety. This application also claims the benefit of each of U.S. Provisional Patent Application No. 63/781,405, filed Apr. 1, 2025, and U.S. Provisional Patent Application No. 63/946,307, filed Dec. 22, 2025, the contents of each which is hereby incorporated by reference in its entirety.

### FIELD OF THE INVENTION

The claimed invention relates to surgical devices, and more specifically to an epicardial clip assembly.

### BACKGROUND

Improved technology is needed to further optimize left atrial appendage exclusion to reduce the risk of systemic embolic events and to optimize heart healing. Embolic events involve the movement of solid materials, often blood clots, also called thrombi, with blood flow down a progressively narrowing blood vessel until they reach a point within the blood vessel that is too narrow to allow their continued passage. Blood flow distal to the obstruction can be severely restricted, leading to ischemic injury and irreparable harm, if untreated. Due to its size, shape, interior contours, and other factors, the left atrial appendage can be a major source of thromboembolism in the systemic circulation (“left heart”). Stroke is a major cause of tragic disability and death across the world. Ischemic stroke in the brain is defined as a cerebral vascular accident that causes significant compromise to the flow of oxygenated blood to the brain, leading to a permanent injury. In addition to injuries to the brain, thromboembolic events can occur in many other critical organs, such as the heart, bowel, kidneys, etc. Currently available pharmacologic and mechanical means to protect patients from such LAA thromboembolism risk are all suboptimal. An absorbable epicardial clip can help fulfill this unmet clinical need.

Located within the pericardial sac, close to the left ventricle, the left atrial appendage (LAA) is a blind outpouching on the left atrium. With reference to FIG. 1A, the LAA 200 is typically a finger-shaped, long, hollow or tubular structure, often with hooks or crenulation features at the more distal end, and a narrow junction at the proximal end that communicates with the left atrium 202. The innermost layer of the LAA is an endothelial lining with a complex textured wall of variable thickness and density between the outermost epicardial layer.

The LAA's irregular shape is often loosely classified by the appearance of its lobes. (e.g., shapes: cauliflower, cactus, windsock, chicken wing, etc.). In a healthy patient, the LAA acts as a contractile reservoir, receiving blood and emptying, thereby washing out the LAA to prevent blood from pooling or stagnating (i.e., often referred to as blood stasis). Specialized tissue in the walls of the LAA are also known to have neurohormonal effects. When normal blood flow within the heart is disrupted (e.g., with atrial fibrillation or mitral valve disease), LAA filling, emptying, and blood velocities can be altered, leading to thrombus formation within the LAA secondary to blood stasis. For example, it has been shown that patients with atrial fibrillation, having no treatment, being undertreated, or being noncompliant with their treatment on oral anticoagulation experience a high incidence of stroke. Chronic oral anticoagulation, often Coumadin, reduces the higher risk of stroke, but increases the risk of significant hemorrhagic events. Currently available pharmacologic agents reduce the risk of thrombus formation by giving the patient a bleeding disorder.

Many questions remain regarding the long-term effects and risks of permanent mechanical LAA occlusive devices. Devices that are passed through the vascular system. (i.e., percutaneous) to be placed on the inside of the heart (i.e., endocardial) have a risk of causing stroke, bleeding, or hypercoagulative states. Endocardial devices must be permanent, and therefore alter the shape of the LAA throughout the life of the patient. Permanent epicardial clips do not have the risks of access through the circulating blood for their placement, but require surgery, with its known complications and risks. Permanent epicardial clips also present the long-term risk of leaving a rigid, structural foreign body element forever attached to the left atrium, with the potential of not allowing the heart to remodel based on the hemodynamic and evolving circulatory needs of the patient.

As patients age and encounter additional cardiac diseases, it appears preferable to have the left atrium unencumbered by an attached rigid foreign material. An epicardial clip, which offers the ability to absorb after achieving its leak-free obliteration of the LAA may offer an improved physiologic and functional heart over time. The therapeutic options for patients at higher risk for LAA-mediated thromboembolic events include heart rate and rhythm control, anticoagulation therapy, cardioversion, heart tissue ablation, and, during the past several decades, the mechanical isolation, by closure or exclusion, of the LAA, with or without its excision. Suturing or stapling the LAA closed can be effective, but both typically require excision of the LAA to enhance healing. Both sutures and staples are required to penetrate the tissue of the LAA and can present a significant risk for bleeding and other complications that reduce their acceptance rate by healthcare practitioners. Endocardial mechanical devices are growing in clinical utilization, but their high cost and substantial risks, along with their need to be permanently affixed to the beating heart of patients, may limit their applicability across populations, especially in less developed medical facilities. To date, all epicardial clips include permanent mechanical structural elements attached to the heart, with yet unknown consequences.

### BRIEF DESCRIPTION OF THE DRAWINGS

FIG. 1A is a cross-sectional view of a heart;

FIG. 1B is a detailed view of the left atrium of the heart following the application of an epicardial clip to the LAA to occlude the LAA;

FIG. 1C is the detailed view of the left atrium approximately two months after the application of the epicardial clip to the LAA;

FIG. 1D is the detailed view of the left atrium approximately one year after the application of the epicardial clip to the LAA, wherein the applied epicardial clip is an absorbable epicardial clip;

FIG. 1E is the detailed view of the left atrium approximately one year after the application of the epicardial clip to the LAA, wherein the applied epicardial clip includes; permanent mechanical structural elements;

FIGS. 2A to 2G are various views of an embodiment of an absorbable clip body assembly;

FIG. 3A is a detailed side view of a hinge portion of the embodiment of the clip body assembly of FIG. 2A;

FIG. 3B is a detailed top view of the hinge portion of the embodiment of the clip body assembly of FIG. 2A;

FIG. 4A is a cross-sectional view of the embodiment of the clip body assembly taken along section line 4A-4A of FIG. 2A;

FIGS. 4B and 4C are cross-sectional views of the embodiment of the clip body assembly taken along section line 4B-4B of FIG. 2A;

FIG. 4D is a cross-sectional view of the embodiment of the clip body assembly taken along section line 4D-4D of FIG. 2A;

FIG. 5A is a cross-sectional view of the embodiment of the clip body assembly taken along section line 5A-5A of FIG. 3B;

FIG. 5B is a cross-sectional view of the embodiment of the clip body assembly taken along section line 5B-5B of FIG. 3B;

FIGS. 6A and 6B are various views of embodiments of an absorbable epicardial clip assembly that includes the embodiment of the clip body assembly in a closed or engaged position;

FIG. 6C is a side view of the embodiment of the clip body assembly in an open or disengaged position;

FIG. 7 is a cross-sectional view of the first arm body of the embodiment of the clip body assembly taken along section line 4A-4A of FIG. 2A showing a portion of suture injection molded into the first arm body;

FIGS. 8A to 8D are various views of a further embodiment of a clip body assembly;

FIG. 9 is a cross-sectional view of the embodiment of the clip body assembly taken along section line 9-9 of FIG. 8B;

FIGS. 10A and 10B are cross-sectional views of the embodiment of the clip body assembly taken along section line 10A-10A of FIG. 8A;

FIG. 10C is a cross-sectional view of the embodiment of the clip body assembly taken along section line 10C-10C of FIG. 8A;

FIGS. 11A to 11C are various views of a further embodiment of a clip body assembly;

FIG. 12 is a cross-sectional view of the embodiment of the clip body assembly taken along section line 12-12 of FIG. 11A;

FIG. 13A is a cross-sectional view of the embodiment of the clip body assembly taken along section line 13A-13A of FIG. 11A;

FIG. 13B is a cross-sectional view of the embodiment of the clip body assembly taken along section line 13B-13B of FIG. 11A;

FIG. 14 is a perspective view of the cross-sectional view of FIG. 13A;

FIGS. 15A to 15C are various views of a further embodiment of a clip body assembly;

FIG. 16 is a cross-sectional view of the embodiment of the clip body assembly taken along section line 16-16 of FIG. 15A;

FIGS. 17A and 17B are cross-sectional views of the embodiment of the clip body assembly taken along section line 17A-17A of FIG. 15A;

FIG. 17C is a cross-sectional view of the embodiment of the clip body assembly taken along section line 17C-17C of FIG. 8A;

FIG. 18 is a side view of embodiments of an absorbable epicardial clip assembly that includes an embodiment of the clip body assembly in a closed or engaged position;

FIG. 19A is a cross-sectional views of the embodiment of the absorbable epicardial clip assembly taken along a section line corresponding to section line 4A-4A of FIG. 2A;

FIG. 19B is a cross-sectional view of the embodiment of the absorbable epicardial clip assembly taken along section line 19B-19B of FIG. 18;

FIGS. 20A and 20B are perspective views of an embodiment of a first spine portion of the absorbable epicardial clip assembly of FIG. 18;

FIGS. 21A to 21G are various views of a further embodiment of a clip body assembly;

FIGS. 22A to 22D are various views of a first arm assembly of the embodiment of the clip body assembly of FIGS. 21A to 21G;

FIG. 23 is a cross-sectional view of the embodiment of the clip body assembly taken along section line 23-23 of FIG. 21B;

FIG. 24 is a cross-sectional view of the embodiment of the first arm assembly taken along section line 24-24 of FIG. 22B;

FIG. 25 is a cross-sectional view of the embodiment of the clip body assembly taken along section line 25-25 of FIG. 22B;

FIG. 26 is a detail of an end portion of the cross-sectional view of the embodiment of the first arm assembly of FIG. 24;

FIG. 27 is a perspective detail of the end portion of the embodiment of the first arm assembly of FIG. 22D;

FIGS. 28A to 28F are various views of a first spine portion of the first arm assembly of the embodiment of the clip body assembly of FIGS. 21A to 21G; and

FIGS. 29 and 30 are top and perspective views, respectively, of an end of the first spine portion of FIGS. 21A to 21G.

### DETAILED DESCRIPTION OF THE INVENTION

As more and more research affirms the effectiveness of LAA exclusion devices, studies are ongoing to evaluate the appropriateness of use of such permanent devices in patients undergoing cardiac surgery, but at lower risk of LAA-mediated thromboembolism (e.g., heart surgery patients without atrial fibrillation). Interestingly, recent publications indicate that patients without atrial fibrillation undergoing heart surgery who receive permanent epicardial clips appear to be at higher risk of developing postoperative atrial fibrillation. We hypothesize that an epicardial clip that facilitates leak-free LAA obliteration, but structurally goes away over time, may offer a more optimized solution.

An absorbable epicardial clip that can achieve the excellent results demonstrated with our current LAX™ Delivery Device and LAX™ nonabsorbable Epicardial Clip may offer substantial acute and long-term clinical advantages. Careful testing and analysis, including hundreds of tests in a wide variety of hearts (i.e., human, canine, caprine, porcine, ovine) and with sophisticated digital testing equipment, demonstrate the parameters preferred for optimized LAA obliteration and closure site healing. A properly closed epicardial clip applies pressure to the contacted compressed tissue to induce ischemic necrosis along the closure line. It also cuts off the blood flow distal to the clip, thereby leading to LAA tissue death and reabsorption. Studies are underway to demonstrate that the absorbable LAA clip is comparably effective throughout healing and will leave little or no mechanical residuals to impede long-term heart remodeling. In addition, the neuroendocrine benefits of the LAA tissue obliteration (for example, including reported lower blood pressure) post-LAA clipping should be realized. Epicardial clips have also been shown to electrically isolate the LAA from contributing to future foci of aberrant rhythms. Blood trapped within the closed LAA will also be absorbed, and the LAA and blood will be replaced by fibrotic tissue.

The surgical materials anticipated for inclusion in this absorbable epicardial clip can be selected based on their physical properties and features, along with how they perform over time in the natural healing environment on the beating heart of the patient. Importantly, the structural features of the absorbable epicardial clip are designed to cooperate with the material properties of the selected materials to maintain a desired pressure profile across the compressed tissue of the LAA when the absorbable epicardial clip is applied to the LAA.

General characteristics of the biomaterials for use in this invention will be selected to optimize material strength, functionality, and stability, while performing the occlusive role during healing. Other general characteristics consider their biocompatibility and biodegradeability, their interactions with selected materials, along with healing tissues and their ability to be handled and sterilized. The absorbable clip jaws need to remain sufficiently stable and maintain their tissue gripping functions throughout at least several weeks of healing to ensure that the compressed tissues remain hermetically sealed until the LAA and the absorbable epicardial clip are effectively absorbed by the body. Ischemic necrosis and scar tissue ingrowth along the occlusive line of the LAA due to adequately and stably compressed capillary beds of the LAA is also achieved by the clip jaws. Examples of injection moldable polymers currently under investigation for this application that can be designed to maintain ample jaw beam strength are polydioxanone (PDO), polyglycolic acid (PGA), and poly-L-lactic acid (PLLA). The absorbable coverings over the jaws should enhance the tissue engagement and inhibit clip migration while safely absorbing over time. For example, coverings made from synthetic absorbable meshes incorporating polyglactin, polyglycolic acid, and/or polyglycolic acid/trimethylene carbonate could be appropriate options for the clip jaws. The sutures and fasteners may be nonabsorbable or absorbable, since previous penetrating suture-or staple-mediated methods of LAA closure have demonstrated that such small, non-structurally impairing materials allow for heart tissue healing and remodeling without requiring significant accommodation.

Included materials must be tailored to accommodate healthy healing rates with added safety factors to always provide ample closure security, even in patients with suboptimal health or healing characteristics or those who suffer from non-clip-related postoperative complications. For economic and sustainability purposes, included materials should preferably offer manufacturing options consistent with reliable processes such as injection molding or textile weaving from already commercially available, clinically proven biomaterials. Such preferred biomaterial candidates typically degrade in the human body over a known time period with a loss of their structural integrity at a controlled rate. The most common injection moldable and textile materials currently in use naturally degrade at known rates in the body over time, primarily by hydrolysis of their ester bonds. Their breakdown products are also biocompatible and benign; they are typically excreted by urine or respiratory gases.

To some degree, all surgical polymers are recognized as foreign materials within the body, to which the host reacts with an immune response, leading to subsequent inflammation as part of healthy healing. Biomaterials suitable for this application may be copolymers or blends or weaves of multiple polymers. Materials selected can be chosen to enhance tissue reactivity. For example, a jaw sheath material can enable enhanced tissue ingrowth prior to its absorption. Selected materials may also be coated or impregnated with biocompatible materials to, for example, improve biocompatibility and/or provide antibacterial properties.

The design intent of this invention is to deliver a safe and easily placed structurally transient epicardial clip that eliminates the risk of the LAA as a source of thromboembolism, along with providing potentially improved neuroendocrine function and electrical isolation. Thus, the absorbable clip is intended to eliminate the need for potentially dangerous drugs, along with the undesired presence of a permanent foreign body attached to the left atrium.

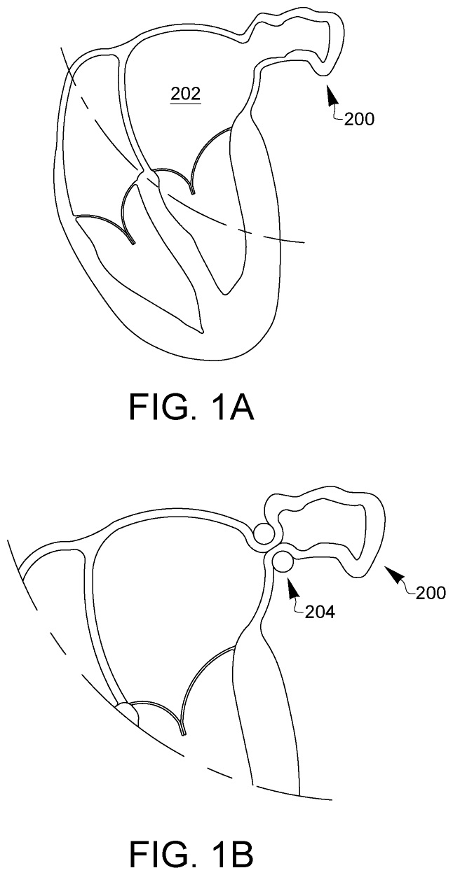

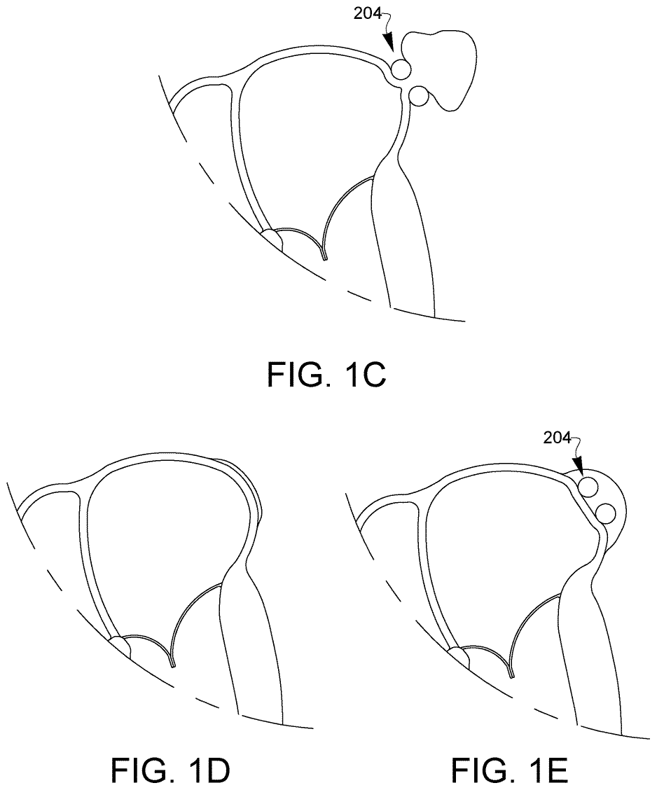

FIGS. 1A to 1D are provided to better illustrate the distinction between an absorbable epicardial clip that includes structurally transient material and an epicardial clip that includes permanent mechanical structural elements. With reference to FIG. 1B, and as previously explained, the properly closed epicardial clip 204 applies pressure to the contacted compressed tissue of the LAA to induce ischemic necrosis along the closure line, while also cutting off the blood flow distal to the clip 204, thereby leading to LAA tissue death. This tissue death is depicted in FIG. 1C, which illustrates the LAA approximately two months after the application of the clip 204. If the epicardial clip 204 is an absorbable epicardial clip, such as an embodiment of the epicardial clip assembly 10 described below, the LAA reabsorbs and leaves little or no mechanical residuals to impede long-term heart remodeling, as illustrated in FIG. 1D, which illustrates the LAA approximately one year after the application of the clip 204. By contrast, if the epicardial clip 204 includes permanent mechanical structural elements, these mechanical structural elements remain attached to the heart, as illustrated in FIG. 1E, which illustrates such a non-absorbable epicardial clip 204 secured to the LAA approximately one year after the application of the clip 204.

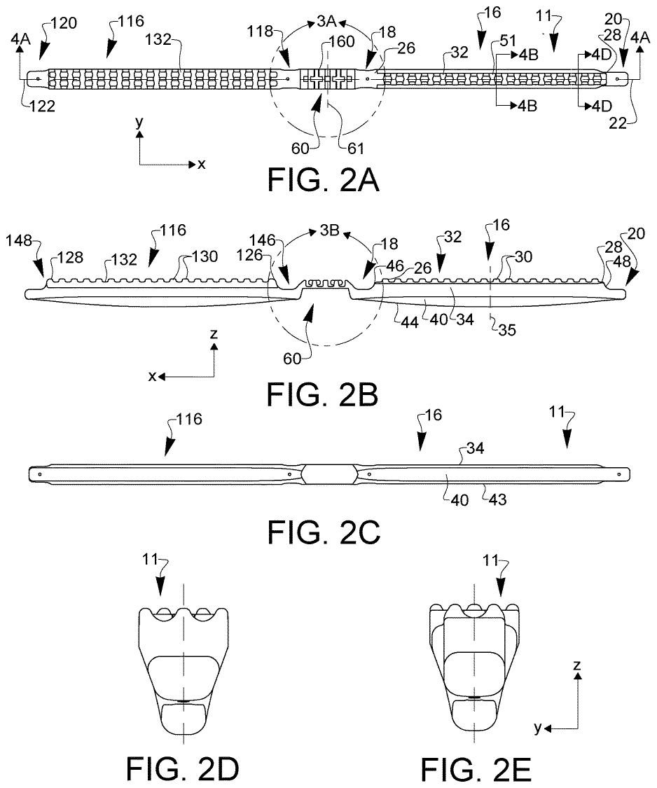

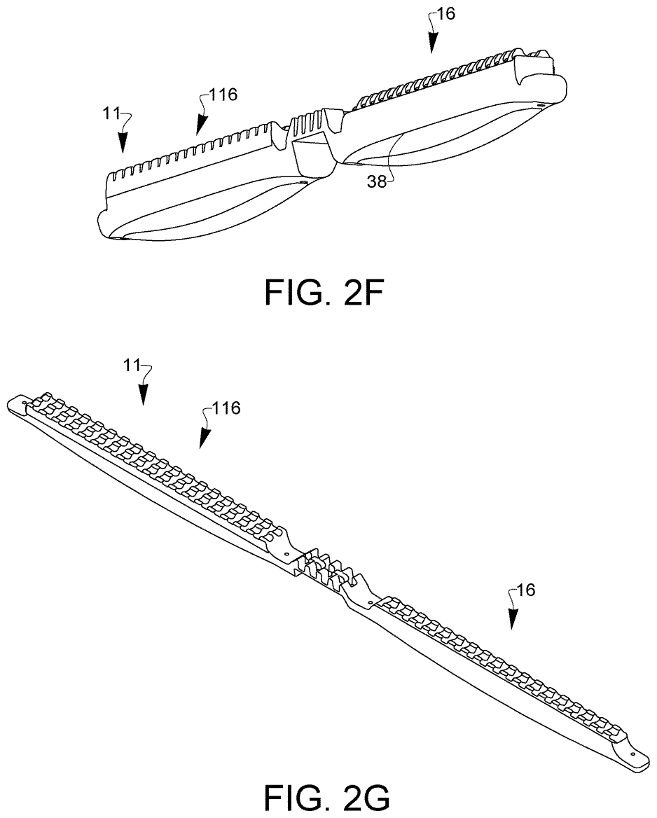

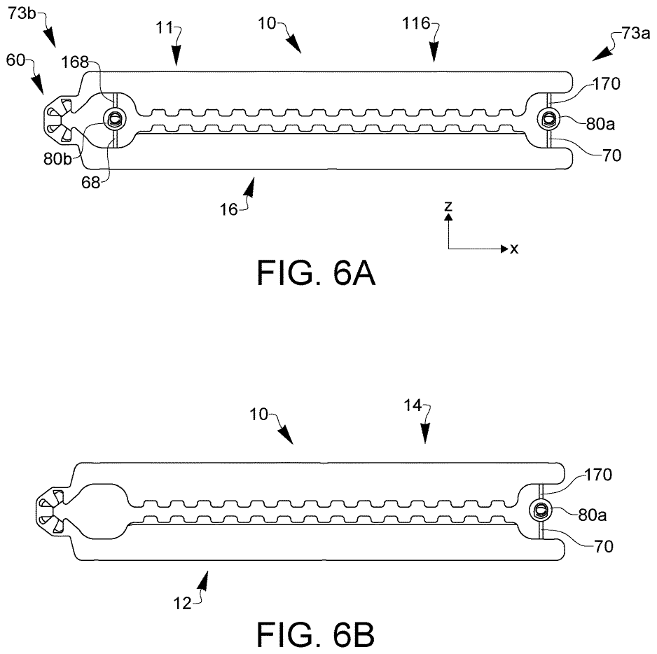

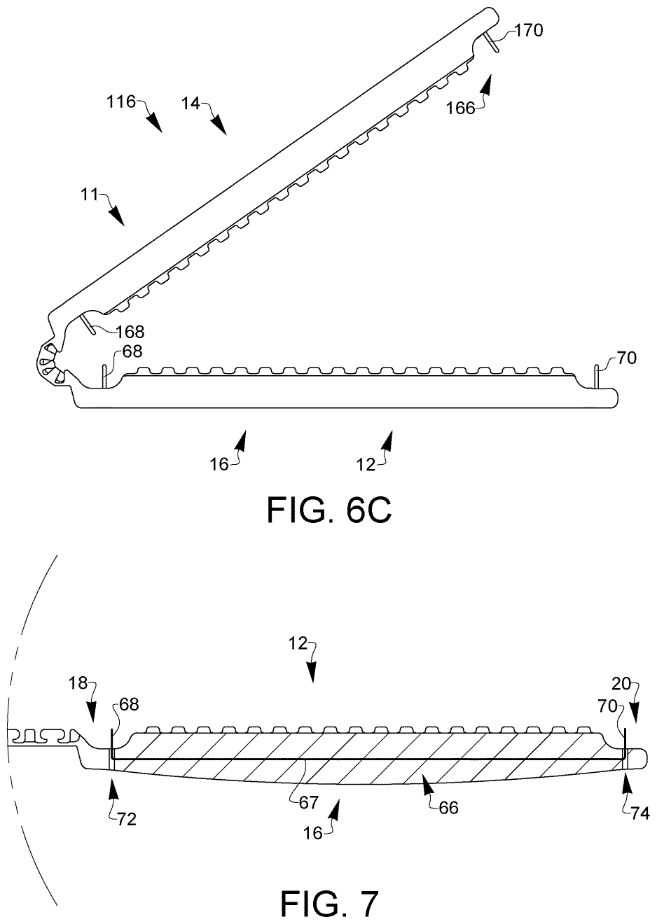

Turning to FIG. 6A, an embodiment of such an absorbable epicardial clip assembly 10 includes a clip body assembly 11, which is illustrated in FIGS. 2A to 2G. With reference to FIG. 2A, the clip body assembly 11 includes a first arm body 16 coupled to a second arm body 116. The first arm body 16 may be coupled to the second arm body 116 in any suitable manner, such as pivotably coupled to the second arm body 116 by a hinge portion 60, and the hinge portion 60 may be a living hinge. The clip body assembly 11 may be configured to be applied to the LAA, and in some embodiments, a portion of the LAA may be placed between a portion of the first arm body 16 and a portion of the second arm body 116. As will be described in more detail below, one or more portions of suture may be coupled to one or both end portions of each of the first arm body 16 and the second arm body 116, and end portions of the suture may be coupled, such as with a crimpable fastener, to form the absorbable epicardial clip assembly 10 that secures the first arm body 16 and the second arm body 116 in an engaged position occluding a portion of the LAA. The use of one or two fasteners, such as crimpable fasteners, to secure the clip body assembly 11 in the engaged position provides a closing force to the compressed tissues of the LAA that will not significantly reduce before the permanent occlusion of the blood flow between the left atrium and the LAA with no communication or leakage, and in some embodiments, until the LAA and the clip body assembly 11 are effectively absorbed by the body. In some embodiments, the clip body assembly 11 may take on moisture when secured to the LAA and may experience a geometric swelling effect that may be caused, at least in part, by the process of hydrolysis. This swelling or overall volume increase of the clip body assembly 11 results in a corresponding increase in the closing force applied to the portion of the LAA positioned between a portion of the first arm body 16 and a portion of the second arm body 116. For example, over a period of 10 hours, the closing force may increase by approximately 19% (approximately 160 mmHg to approximately 190 mmHg), and over a period of 50 hours, the closing force may increase by approximately 25% (approximately 160 mmHg to approximately 200 mmHg). In some embodiments, the closing force may increase in a range of 5% to 40%. This increase may occur substantially immediately or over a short first period of time, such as approximately 1 hour to approximately 10 hours. This increase in closing force may gradually increase for the first period of time, and may remain constant or substantially constant for a second period of time that is greater or substantially greater than the first period of time. In some embodiments, the second period of time may end upon the degradation of the structural integrity of the clip body assembly 11. This increase in closing force while the first arm body 16 and the second arm body 116 are in the engaged position leads to more rapid ischemic necrosis and scar tissue ingrowth along the occlusive line of the LAA by providing additional compression to the capillary beds of the LAA.

In some embodiments, the cross-sectional shapes of the first arm body 16 and the second arm body 116 may vary along the longitudinal axis of the first arm body 16 and the second arm body 116 and may be designed to optimize the pressure profile (or achieve a desired pressure profile) of the first arm body 16 and the second arm body 116 on the tissue of the LAA when it is occluding the portion of the LAA. In some embodiments, a first cross-sectional shape (or a first plurality of cross-sectional shapes) may be selected for optimal pressure for an LAA having a first thickness and a second cross-sectional shape (or a second plurality of cross-sectional shapes) may be selected for optimal pressure for an LAA having a second thickness. Additional cross-sectional shapes may be provided for further LAA thicknesses.

As will also be described in more detail below, the clip body assembly 11 may be a single, unitary part made of an injection molded material that is bioabsorbable or biocompatible, such as any of the materials described above. In some embodiments, the one or more portions of suture and the fasteners may also be made from bioabsorbable material.

So configured, the clip body assembly 11 (or the absorbable epicardial clip assembly 10) may provide immediate and permanent occlusion of the blood flow between the left atrium and the LAA with no communication or leakage. In the engaged position, the clip body assembly 11 provides sufficient and comparable pressure across the apposed tissue planes of the LAA to ensure accomplishment of ischemic necrosis to facilitate subsequent distal tissue absorption and tissue healing and remodeling within the compression zone. While providing this optimal pressure, the clip body assembly 11 prevents excessive forces within the compression zone that transect the compressed tissue. Secured as described, the clip body assembly 11 (or the absorbable epicardial clip assembly 10) maintains its holding location long enough to enable permanent healing across the compression area without leakage during a period of about two weeks to three months, and the clip body assembly 11 remains biocompatible throughout the process. In some embodiments, the clip body assembly 11 maintains its structural integrity to provide occlusion and ischemia during a period of not less than two weeks and not more than two years. For example, the clip body assembly 11 may maintain its structural integrity to provide occlusion and ischemia during a period of approximately one year. After the permanent healing and exclusion of the distal LAA, the clip body assembly 11 will lose its physical structure by absorption so that it no longer influences the remodeling of the LAA by its physical presence. Following the degradation of the structural integrity of the clip body assembly 11 as described, two effects associated with the presence of a foreign body are improved. The first is that the absorbed clip body assembly 11 has a minimized effect on the natural healing topography and remodeling of the heart when compared to a permanent foreign body. Second, the movement of the heart is not impeded by the absorbed clip body assembly 11, as opposed to the interference to the movement of the heart caused by a permanent foreign body.

Further, the streamlined design of the clip body assembly 11 (or the absorbable epicardial clip assembly 10) may allow a surgeon to precisely position the clip body assembly 11 (or the absorbable epicardial clip assembly 10) at the junction of the left atrium and the LAA for optimal occlusion of the LAA. Such positioning, coupled with the crimpable fasteners securing suture that is partially embedded in portions of the clip body assembly 11, prevents the clip body assembly 11 from displacing relative to its application position on the LAA. Further, if the clip body assembly 10 is positioned incorrectly on a first attempt during a procedure, the suture may be easily severed to remove the clip body assembly 11, and the clip body assembly 11 (or another clip body assembly 11) may be secured to the LAA in the desired position.

Turning to the clip body assembly 11 in more detail, an embodiment of the clip body assembly 11 is illustrated in FIGS. 2A to 2G in a linear orientation in which it may be manufactured using an injection molding process. However, for use, the first arm body 16 may be pivoted about the hinge portion 60 into the orientation illustrated in FIGS. 6A to 6C for use in a procedure. As illustrated in FIGS. 2A and 2B, the first arm body 16 may be elongated and may extend from a first end 18 to a second end 20 along a first arm body axis 22. The first arm body 16 may include an engagement surface 32 along all or a portion of the first arm body 16, and the engagement surface 32 may be configured to engage a portion of the LAA when the clip body assembly 11 is in the closed or engaged position. The engagement surface 32 may extend from a first end 26 to a second end 28 along the first body axis 22. The first end 26 may be inwardly offset from the first end 18 of the first arm body 16, and the second end 28 may be inwardly offset from the second end 20 of the first arm body 16. The engagement surface 32 may have any suitable shape or combination of shapes, such as a planar or substantially planar shape that may be parallel to the X-Y plane of the reference coordinate system of FIG. 2A. However, all or one or more portions of the engagement surface 32 may be curved, contoured, or angled. A plurality of projections 30 may extend from the engagement surface 32 of the first arm body 16 to provide a secure grip or bite on the portion of the LAA when the clip body assembly 11 is in the closed position.

The first arm body 16 may also include a first notch edge portion 46 that may extend along a portion of the first arm body 16 from the first end 26 of the engagement surface 32 to the first end 18 of the first arm body 16, and the first notch edge portion 46 may have a concave, arcuate, or semi-circular shape when viewed along the Y-axis of the reference coordinate system of FIGS. 2A and 2B. A second notch edge portion 48 may extend along a portion of the first arm body 16 from the second end 28 of the engagement surface 32 to the second end 20 of the first arm body 16, and the second notch edge portion 48 may have a concave, semi-concave, or semi-circular shape when viewed along the Y-axis of the reference coordinate system of FIG. 2A.

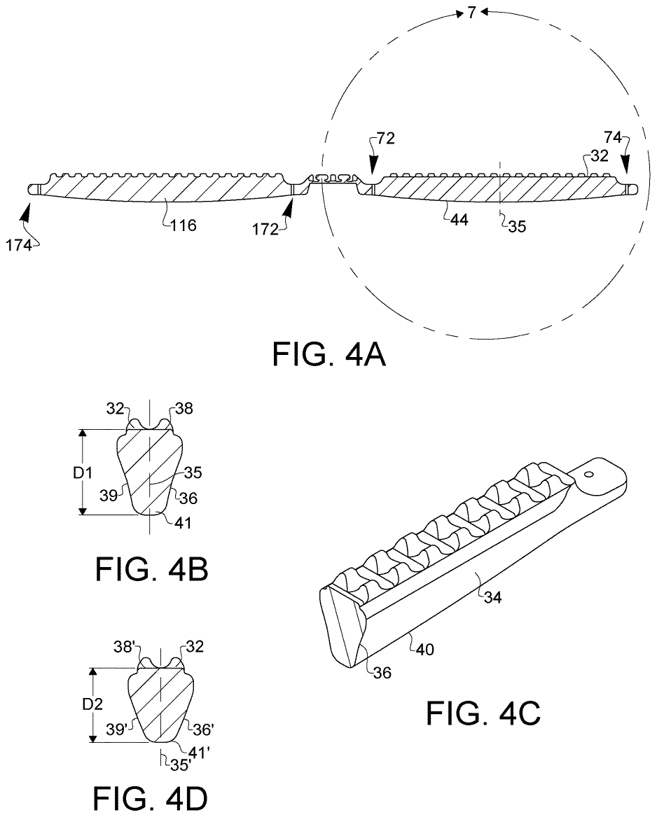

The first arm body 16 may have any suitable cross-sectional shape when viewed normal to the X axis of the reference coordinate system of FIG. 2A. For example, the first arm body 16 may have a rectangular shape, a generally rectangular shape, a generally triangular shape, or a generally semicircular shape along all or a portion of the first arm body axis 22, such as a portion along the length of the engagement surface 32. In other embodiments, the cross-sectional shape may vary along the first arm body axis 22. For example, in the cross-sectional view of FIG. 4B, which is taken along the section line 4B-4B of FIG. 2A that is located at a midpoint 51 (which is midway between the first end 26 and the second end 28 of the engagement surface 32 and may be midway between the first end 18 and the second end 20 of the first arm body 16), the first arm body 16 may include a first lateral wall 34 (see FIG. 4C) that may may be defined by a first lateral edge 36. The first lateral edge 36 may extend from a first end to a second end, and the first end may be at or adjacent to an end of an edge 38 of the engagement surface 32 and may be coupled to the end of the edge 38 of the engagement surface by a rounded or contoured edge portion. The first lateral edge 36 may be linear or substantially linear, and may form an acute angle with an axis 35 that is parallel to the Z-axis of the reference coordinate system of FIG. 2A (and that extends through the first body axis 22) such that the first lateral edge 36 diverges from the axis 35 as the first lateral edge 36 extends from the second end towards the first end. In some embodiments, the first lateral edge 36 may be contoured or arcuate such that the first lateral edge 36 has a shallow concave shape.

Still referring to FIG. 4B, the first arm body 16 may include a second lateral wall 43 (see FIG. 2C) that may may be defined by a second lateral edge 39 in the cross-sectional view. The second lateral edge 39 may have any suitable shape or combination of shapes and may differ in shape from the first lateral edge 36. However, the second lateral edge 39 may be a mirror image of the first lateral edge 36 taken about the axis 35. That is, the second lateral edge 39 may extend from a first end to a second end, and the first end may be at or adjacent to an opposite end of the edge 38 of the engagement surface 32 and may be coupled to the opposite end of the edge 38 of the engagement surface 32 by a rounded or contoured edge portion. The second lateral edge 39 may be linear or substantially linear, and may form the acute angle (or any other acute angle) with the axis 35 such that the second lateral edge 39 diverges from the axis 35 as the second lateral edge 39 extends from the second end towards the first end. In some embodiments, the second lateral edge 39 may be contoured or arcuate such that the second lateral edge 39 has a shallow concave shape.

A contoured or rounded bottom portion 40 (see FIG. 2C) may be defined by a bottom edge 41 when viewed in cross-section, and the bottom edge 41 may extend from the second end of the first lateral edge 36 to the second end of the second lateral edge 39, and the bottom edge 41 may be symmetrical about the axis 35. In some embodiments, the bottom edge 41 may have a semicircular or arcuate shape. A point where the bottom edge 41 intersects the axis 35 may extend a first distance D1 from the engagement surface 32.

The cross-sectional shape of the first arm body 16 may vary from the midpoint 51 (along section line 4B-4B of FIG. 2A) towards the second end 28 of the engagement surface 32. In particular, the distance between the point where the bottom edge 41 intersects an axis (which is parallel to the axis 35 and which extends along the cross-sectional plane) and the engagement surface 32 may decrease from the midpoint 51 towards the second end 28 of the engagement surface 32 when taken along the first body axis 22. For example, in the cross-sectional view of FIG. 4D, which is taken along the section line 4D-4D of FIG. 2A that is located adjacent to, but offset from, the second end 28 of the engagement surface 32, a point where the bottom edge 41′ intersects an axis 35′ (that may be parallel to the axis 35 and which extends along the cross-sectional plane indicated by section line 4D-4D) may extend a second distance D2 from the edge 38′ of the engagement surface 32, and this second distance D2 may be less than the first distance D1 of FIG. 4B. In some embodiments, the bottom edge 41′ of the cross-section of FIG. 4D may have the same or substantially the same shape and dimensions as the bottom edge 41 of the cross-section of FIG. 4B, but the first lateral edge 36′ and the second lateral edge 39′ may each be shorter than the first lateral edge 36 and the second lateral edge 39 of FIG. 4B. That is, a distance between the edge 38′ of engagement surface and the second end of the first lateral edge 36′ of FIG. 4D is less than a distance between the edge of engagement surface 32 and the second end of the first lateral edge 36 of FIG. 4B. Similarly, a distance between the opposite edge of engagement surface and the second end of the second lateral edge 39′ of FIG. 4D is less than a distance between the opposite edge of engagement surface 32 and the second end of the second lateral edge 39 of FIG. 4B.

The cross-sectional shape of the first arm body 16 may also vary from the midpoint 51 towards the first end 26 of the engagement surface 32. In some embodiments, the cross-sectional shape of the first arm body 16 from the midpoint 51 of the engagement surface 32 to the second end 28 of the engagement surface 32 may be a mirror image of the cross-sectional shape of the first arm body 16 from the midpoint 51 to the first end 26 of the engagement surface 32, and the mirror plane or axis may extend through or along section line 4B-4B of FIG. 2A. So configured, when the first arm body 16 is viewed in the cross-sectional view of FIG. 4A, which is taken along section line 4A-4A of FIG. 2A, a bottom edge 44 of the bottom portion 40 may form a cambered, arced, or semi-circular shape from the first end 26 to the second end 28 of the engagement surface 32 (and/or to the first end 18 and second end 20 of the first arm body 16), and this shape may be symmetrical about the axis 35.

As illustrated in FIG. 2A, the clip body assembly 11 also includes the second arm body 116, and the second arm body 116 may be elongated and may extend from a first end 118 to a second end 120 along a second arm body axis 122. The second arm body 116 may be s identical (or substantially identical) to, but a mirror image of, the first arm body 16 and the structure of the second arm body 116 may include any or all of the structural elements described in terms of the first arm body 16. In some embodiments, a first notch edge portion 146 may extend along a portion of the second arm body 116 from the first end 126 of the engagement surface 132 to the first end 118 of the second arm body 116, and the first notch edge portion 146 may have a concave, arcuate, or semi-circular shape when viewed along the Y-axis of the reference coordinate system of FIGS. 2A and 2B. A second notch edge portion 148 may extend along a portion of the second arm body 116 from the second end 128 of the engagement surface 132 to the second end 120 of the second arm body 116, and the second notch edge portion 148 may have a concave, semi-concave, or semi-circular shape when viewed along the Y-axis of the reference coordinate system of FIG. 2A.

The cross-sectional shape of the first arm body 16 (and the second arm body 116) may be configured to achieve a desired stiffness that results in an evenly distributed pressure (or evenly distributed forces) across a portion of the LAA when the LAA is disposed between all or a portion of the engagement surface 32 of the first arm body 16 and all or a portion of the engagement surface 128 of the second arm body 116 when the clip body assembly 11 is secured in the closed or engaged position of FIG. 4A (or 4B). In some embodiments, a first cross-sectional shape may be selected for optimal pressure for an LAA having a first thickness. Subsequently disclosed cross-sectional shapes will provide different stiffnesses or flexibilities across the length of that embodiment of the first arm body (and the second arm body) that may result in different pressures, or may also achieve evenly-distributed pressure across the LAA when the corresponding clip body assembly is the engaged position. For example, each of the subsequently disclosed cross-sectional shapes may be provided to maintain an optimized pressure profile across a portion of the LAA having a unique thickness that may be different than the first thickness.

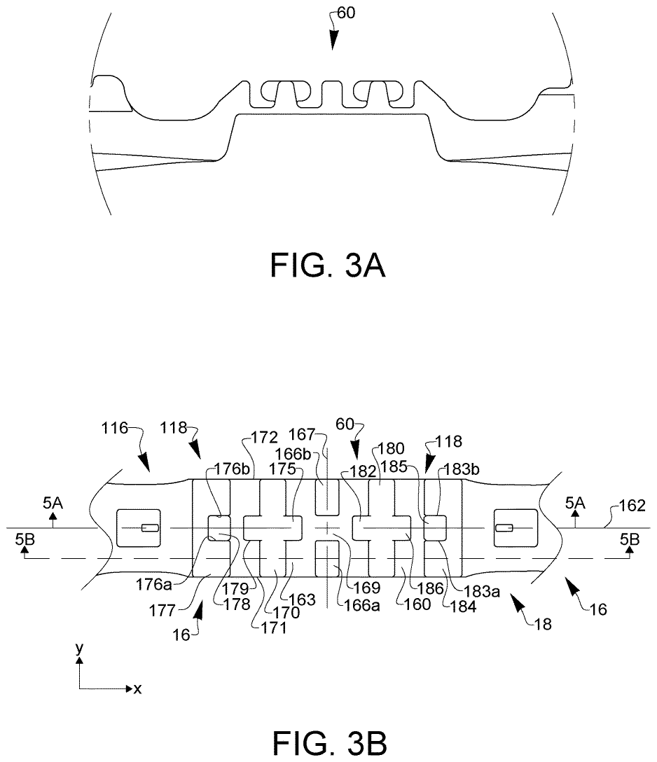

Referring to FIGS. 2A, 2B, and 3A, the clip body assembly 11 may also include the hinge portion 60 that couples the first arm body 16 and the second arm body 116, and the hinge portion 60 may extend from the first end 18 of the first arm body 16 to the first end 118 of the second arm body 116. The hinge portion 60 may be a “living hinge” and may be integrally formed with the first end 18 of the first arm body 16 and the first end 118 of the second arm body 116 such that the hinge portion 60, the first arm body 16, and the second arm body 116 are a single, unitary part, and this part may be manufactured or fabricated as an injection-molded plastic part. The hinge portion 60 may be flexible and may be configured to be bent or to rotate about a pivot axis 61 (see FIG. 2A) that may be parallel to the Y-axis of the reference coordinate system of FIGS. 2A and 2B. In some embodiments, the hinge portion 60 may have one or more features that are configured to prevent rotation of the hinge portion 60 about any axis that is not parallel to the Y-axis.

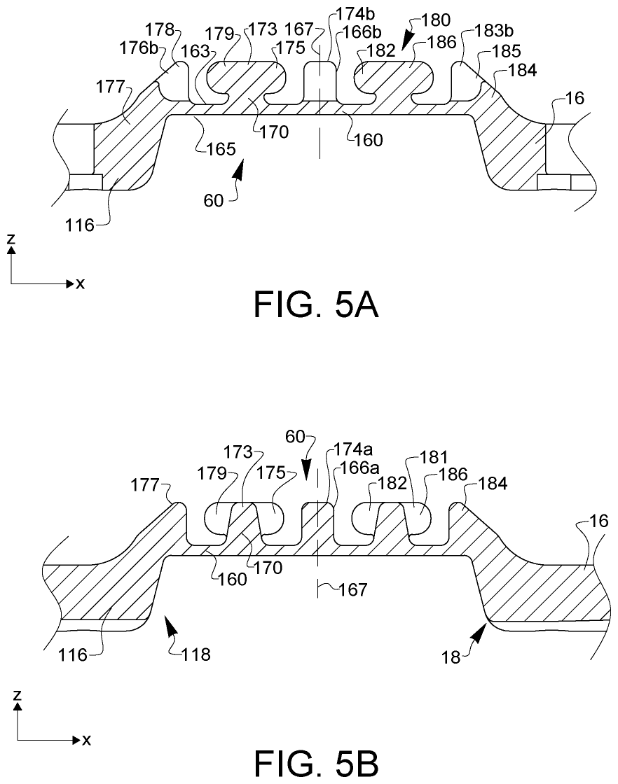

Referring to FIG. 2A, which is a top view of the hinge portion 60 in a linear “flat” configuration, which may be the configuration in which the clip body assembly 11 is manufactured, the hinge portion 60 may include a base member 160 that may extend from the first end 18 of the first arm body 16 to the first end 118 of the second arm body 116 along a base axis 162 (see FIG. 3B) that may be parallel to the X-axis of the reference coordinate system of FIGS. 2A and 3B. The base member 160 may be at least partially defined by a top surface 163 and a bottom surface 165 (illustrated in the cross-sectional view of FIG. 5A) which may each be planar or substantially planar and may be parallel to the X-Y plane of the reference coordinate system of FIG. 2A.

With reference to the detailed top view of the hinge portion 60 of FIG. 3B, the hinge portion 60 may also include a first support protrusion 166a and a second support protrusion 166b that may each upwardly extend from the top surface 163, and the first support protrusion 166a and the second support protrusion 166b may be aligned along, and each symmetrically disposed about, a reference plane 167 that may be parallel to the Y-Z plane of the reference coordinate system of FIG. 3B. An inner lateral wall 168a of the first support protrusion 166a and an inner lateral wall 168bv of the second support protrusion 166b may each be disposed parallel (or substantially parallel) to the X-Z plane of the reference coordinate system of FIG. 3B such that the inner lateral walls 168a, 168b define a center gap 169 disposed along the Y-axis of the reference coordinate system of FIG. 3B.

The hinge portion 60 may also include a first engagement protrusion 170 that may upwardly extend from the top surface 163 of the base member 160 and may laterally extend from a first lateral edge 171 of the base member 160 to a second lateral edge 172 of the base member 160 along (or substantially along) an axis normal to the base axis 162. A top surface 173 of the first engagement protrusion 170 may be parallel to the top surface 163 of the base member 160 and the top surface 173 may be aligned or generally aligned with a top surface 174a, 174b of one or both of the first and second support protrusion 166a, 166b. A first inner tab 175 may extend from a first portion of the top portion of the first engagement protrusion 170 towards the center gap 169 along the base axis 162, and the first inner tab 175 may have a width (parallel to the Y-axis of the reference coordinate system of FIG. 3B) that is less than (or slightly less than) the distance between the inner lateral walls 168a, 168b that defines the center gap 169. Thus, when the absorbable epicardial clip assembly 10 is in the engaged (closed) position illustrated in FIG. 6A, all or a portion of the first inner tab 175 is disposed within a first portion of the center gap 169 defined between the inner lateral walls 168a, 168b. When the absorbable epicardial clip assembly 10 is in the disengaged (open) position illustrated in FIG. 2A or 6C, the first inner tab 175—may be disposed outside of the center gap 169 defined between the inner lateral walls 168a, 168b. However, in some embodiments, the first inner tab 175 may extend a sufficient distance towards the center gap 169 that a portion of the first inner tab 175 may be disposed within the first portion of the center gap 169 defined between the inner lateral walls 168a, 168b in the disengaged or partially disengaged position.

Referring again to FIG. 3B, a first lateral wall 176a and a second lateral wall 176b may be formed in a first end protrusion 177 disposed at or extending from the first end 118 of the second arm body 116, and the first lateral wall 176a and the second lateral wall 176b may at least partially define a first end notch 178 disposed in the first end protrusion 177. The first lateral wall 176a and the second lateral wall 176b may be symmetrically disposed about the base axis 162 and aligned with the corresponding inner lateral walls 168a, 168b defining the center gap 169 such that the first end notch 178 is aligned with the center gap 169.

A first outer tab 179 may extend from a second portion of the top portion of the first engagement protrusion 170 towards the first end notch 178 along the base axis 162, and the first outer tab 179 may have a width (parallel to the Y-axis of the reference coordinate system of FIG. 3B) that is less than (or slightly less than) the distance between the first lateral wall 176a and the second lateral wall 176b that defines the first end notch 178. Thus, when the absorbable epicardial clip assembly 10 is in the engaged (closed) position illustrated in FIG. 6A, all or a portion of the first outer tab 179 is disposed within the first end notch 178 defined between the first lateral wall 176a and the second lateral wall 176b. When the absorbable epicardial clip assembly 10 is in the disengaged (open) position illustrated in FIG. 2A or 6C, the first outer tab 179 may be disposed outside of the first end notch 178 defined between the first lateral wall 176a and the second lateral wall 176b. However, in some embodiments, the first outer tab 179 may extend a sufficient distance towards the first end notch 178 that a portion of the first outer tab 179 may be disposed within a portion of the first end notch 178 defined between the first lateral wall 176a and the second lateral wall 176b in the disengaged or partially disengaged position.

The hinge portion 60 may also include a second engagement protrusion 180 that may be identical or substantially identical to the first engagement protrusion 170, and symmetrically disposed about the reference plane 167 from the first engagement protrusion 170. In particular, the second engagement protrusion 180 may upwardly extend from the top surface 163 of the base member 160 and may laterally extend from the first lateral edge 171 of the base member 160 to the second lateral edge 172 of the base member 160 along (or substantially along) an axis normal to the base axis 162. A top surface 181 of the second engagement protrusion 180-342 may be parallel to the top surface 163 of the base member 160 and the top surface 181 may be aligned or generally aligned with the top surface 174a, 174b of one or both of the first and second support protrusion 166a, 166b. A second inner tab 182 may extend from a first portion of the top portion of the second engagement protrusion 180 towards the center gap 169 along the base axis 162, and the second inner tab 182 may have a width (parallel to the Y-axis of the reference coordinate system of FIG. 3B) that is less than (or slightly less than) the distance between the inner lateral walls 168a, 168b that defines the center gap 169. Thus, when the absorbable epicardial clip assembly 10 is in the engaged (closed) position illustrated in FIG. 6A, all or a portion of the second inner tab 182 is disposed within a second portion of the center gap 169 defined between the inner lateral walls 168a, 168b. When the atrial clip assembly 300 is in the disengaged (open) position illustrated in FIG. 2A or 6C, the second inner tab 182 may be disposed outside of the portion of the center gap 169 defined between the inner lateral walls 168a, 168b. However, in some embodiments, the second inner tab 182 may extend a sufficient distance towards the center gap 169 that a portion of the second inner tab 182 may be disposed within the second portion of the center gap 169 defined between the inner lateral walls 168a, 168b in the disengaged or partially disengaged position.

Referring again to FIG. 3B, a first lateral wall 183a and a second lateral wall 183b may be formed in a second end protrusion 184 disposed at or extending from the first end 18 of the first arm body 16, and the first lateral wall 183a and the second lateral wall 183b may at least partially define a second end notch 185 disposed in the second end protrusion 184. The first lateral wall 183a and the second lateral wall 183b may be symmetrically disposed about the base axis 162 and aligned with the corresponding inner lateral walls 168a, 168b defining the center gap 169 such that the second end notch 185 is aligned with the center gap 169.

A second outer tab 186 may extend from a second portion of the top portion of the second engagement protrusion 180 towards the second end notch 185 along the base axis 162, and the second outer tab 186 may have a width (parallel to the Y-axis of the reference coordinate system of FIG. 3B) that is less than (or slightly less than) the distance between the first lateral wall 183a and the second lateral wall 183b that defines the second end notch 185. Thus, when the absorbable epicardial clip assembly 10 is in the engaged (closed) position illustrated in FIG. 6A, all or a portion of the second outer tab 186 is disposed within all or a portion of the second end notch 185 defined between the first lateral wall 183a and the second lateral wall 183b. When the absorbable epicardial clip assembly 10 is in the disengaged (open) position illustrated in FIG. 2A or 6C, the second outer tab 186 may be disposed outside of the second end notch 185 defined between the first lateral wall 183a and the second lateral wall 183b. However, in some embodiments, the second outer tab 186 may extend a sufficient distance towards the second end notch 185 that a portion of the second outer tab 186 may be disposed within a portion of the second end notch 185 defined between the first lateral wall 183a and the second lateral wall 183b in the disengaged or partially disengaged position.

As previously explained, the hinge portion 60 may be flexible and may be configured to be bent or to rotate about the pivot axis 61 (see FIG. 2A). When or as the absorbable epicardial clip assembly 10 is displaced from the disengaged position illustrated in FIG. 2A or 6C to the engaged position illustrated in FIG. 6A, all or a portion of the first inner tab 175 of the first engagement protrusion 170 is disposed within a first portion of the center gap 169, and all or a portion of the first outer tab 179 of the first engagement protrusion 170 is disposed within the first end notch 178 of the first end protrusion 177. Further, all or a portion of the second inner tab 182 of the second engagement protrusion 180 is disposed within a second portion of the center gap 169 and all or a portion of the second outer tab 186 of the second engagement protrusion 180 is disposed within all or a portion of the second end notch 185 of the second end protrusion 184. Thus, the interaction of the tabs within the center gap 169 and notches 178, 185 maintains or substantially maintains the alignment of the first arm body 16 and the second arm body 116 about a plane parallel to the X-Z plane of the reference coordinate system of FIGS. 2A and 3B such that the absorbable epicardial clip assembly 10 may be pivoted only about the pivot axis 61 that may be parallel to the Y-axis of the reference coordinate system of FIG. 2A, thereby eliminating or reducing twisting or torsional forces on the first arm body 16 and/or the second arm body 116 when the clip body assembly 11 is in the engaged (closed) position illustrated in FIG. 6A, between the engaged (closed) position and the disengaged (open) position, or in the disengaged (open) position illustrated of FIG. 6C.

When the first arm body 16 and the second arm body 116 are configured to be applied to the LAA (for example, by using an introducer device such as that disclosed in in U.S. patent application Ser. No. 18/612,814, filed Mar. 21, 2024, which has been incorporated by reference in its entirety), the first arm body 16 and second arm body 116 are positioned into a disengaged position (illustrated in FIG. 6C) for application and then displaced to the engaged position of FIG. 6A, in which the first end 18 of the first arm body 16 is disposed adjacent or aligned (along the Z-axis of the reference coordinate system of FIG. 6A) with the first end 118 of the first arm body 116 and the second end 20 of the first arm body 16 is disposed adjacent or aligned (along the Z-axis of the reference coordinate system of FIG. 6A) with the second end 120 of the second arm body 116. In this engaged position of the clip body assembly 11, with a portion of the LAA disposed between all or a portion of the engagement surface 32 of the first arm body 16 and all or a portion of the engagement surface 132 of the second arm body 116, the first arm body 16 and the second arm body 116 may be secured together in any suitable manner to apply a desired (e.g., even) pressure across the portion of the LAA to isolate the interior of the LAA. In some embodiments, the first end 68 of the one or more portions of suture 66 of the first arm body 16 may be secured to the first end 168 of the one or more portions of suture 166 of the second arm body 116. In addition, the second end 70 of the one or more portions 66 of suture of the first arm body 16 may be secured to the second end 170 of the one or more portions of suture 166 of the second arm body 116. However, in the embodiment of FIG. 6B, the second end 70 of the one or more portions 66 of suture of the first arm body 16 may be secured to the second end 170 of the one or more portions of suture 166 of the second arm body 116, and the first end 68 of the one or more portions of suture 66 of the first arm body 16 and the first end 168 of the one or more portions of suture 166 of the second arm body 116 may not be included.

In some embodiments, such as the embodiment of FIG. 6A, the second end 70 of the one or more portions 66 of suture of the first arm body 16 may be secured to the second end 170 of the one or more portions of suture 166 of the second arm body 116 by a securing element 73a, and the securing element 73a may be any feature or combination of features that couples the second ends 70, 170. A further securing element 73b may also secure the first end 68 of the one or more portions of suture 66 of the first arm body 16 and the first end 168 of the one or more portions of suture 166 of the second arm body 116. In the embodiment of FIG. 6A, the securing element 73 may be a first crimpable sleeve 80a (illustrated in an uncrimped configuration for clarity) that may be used to secure the first end 68 and the first end 168, and/or a second crimpable sleeve 80b (illustrated in an uncrimped configuration for clarity) may be used to secure the second end 70 and the second end 170. The crimpable sleeves 80a, 80b may be crimped or secured using a COR-KNOT® or COR-KNOT MINI® device manufactured by LSI Solutions® to secure both the first ends 68, 168 and the second ends 70, 170 within the crimped sleeve (not shown). In some embodiments, the crimpable sleeves 80a, 80b may be formed, made, or manufactured from an absorbable material. For example, the crimpable sleeve may be any of the embodiments of a sleeve (or knot) disclosed in U.S. Pat. No. 8,398,680 that is titled “Bioabsorbable Magnesium Knots for Securing Surgical Suture” and that issued on Mar. 19, 2013, the contents of which is incorporated by reference herein in its entirety. In some embodiments, the one or more portions of suture 66 of the first arm body 16 and the one or more portions of suture 166 of the second arm body 116 may also be formed, made, or manufactured from an absorbable material.

The first notch edge portion 46 of the first arm body 16 and the first notch edge portion 146 of the second arm body 116 may cooperate to provide a suitable space or gap for the cylindrical first crimpable sleeve 80a such that the first crimpable sleeve 80a does not contact or interfere with the desired positioning of the first arm body 16 and the second arm body 116 when in the engaged position. Similarly, the second notch edge portion 48 of the first arm body 16 and the second notch edge portion 148 of the second arm body 116 may cooperate to provide a suitable space or gap for the cylindrical second crimpable sleeve 80b such that the second crimpable sleeve 80b does not contact or interfere with the desired positioning of the first arm body 16 and the second arm body 116 when in the engaged position.

The suture may be coupled to the first arm body 16 and the second arm body 116 in any suitable manner. In particular, as illustrated in the cross-sectional view of FIG. 4A, the first arm body 16 may include a first transverse aperture 72 that may extend along the Z-axis through a portion of the first arm body 16 that is between the first end 26 of the engagement surface 32 and the first end 18 of the first arm body 16 (or that is disposed at the first end 18 of the first arm body 16). In addition, the first arm body 16 may include a second transverse aperture 74 that may extend along the Z-axis through a portion of the first arm body 16 that is between the second end 28 of the engagement surface 32 and the second end 20 of the first arm body 16.

In some embodiments, as illustrated in the cross-sectional view of FIG. 7, which is a detail of the cross-section of FIG. 4A, a first arm assembly 12 may include the first arm body 16 that includes or is secured to a length of suture 66, and the length of suture 66 may be inserted molded into the first arm body 16 to secure the length of suture 66 to the first arm body 16. In particular, a first portion 67 of the length of suture 66 may extend through a portion of the first arm body 16 between the first transverse aperture 72 and the second transverse aperture 74. The first portion 68 of the length of suture 66 may be disposed in any orientation along or generally along the X-axis of the reference coordinate system of FIG. 2B. For example, the first portion 68 of the length of suture 66 may be disposed parallel or substantially parallel to the X-axis of the reference coordinate system of FIG. 2B. In other examples, the first portion 68 of the length of suture 66 may be disposed in a tortuous path that is generally parallel to the X-axis of the reference coordinate system of FIG. 2B. The first end 68 of the length of suture 66 may extend through and beyond the first transverse aperture 72, and the second end 70 of the length of suture 66 may extend through and beyond the second transverse aperture 74. The first arm assembly 12 may include the first arm body 16 and the molded (or otherwise secured) length of suture

In addition, a second arm assembly 14 may include the second arm body 116 and a molded (or otherwise secured) second length of suture 166, and the hinge portion 60 may couple the first arm assembly 12 to the second arm assembly 14 as previously described. The second length of suture 166 may be inserted molded into the second arm body 116 to secure the second length of suture 166 to the second arm body 116 in the same manner as described for the length of suture 66 coupled to the first arm body 16. That is, with reference to FIG. 4A, a first portion (not shown) of the second length of suture 166 may extend through a portion of the second arm body 116 between a first transverse aperture 172 and a second transverse aperture 174. With reference to FIG. 6A, the first end 168 of the second length of suture 166 may extend through and beyond the first transverse aperture 172, and the second end 170 of the second length of suture 166 may extend through and beyond the second transverse aperture 174.

So configured, and as illustrated in FIGS. 6A and 6B, the second end 70 of the one or more portions of suture 66 of the first arm assembly 12 may be secured to the second end 170 of the one or more portions of suture 166 of the second arm assembly 14 (and, optionally, the first end 68 of the one or more portions of suture 66 of the first arm assembly 12 may be secured to the first end 168 of the one or more portions of suture 166 of the second arm assembly 14) in the manner described above to secure the clip body assembly 11 to a portion of the LAA.

In other embodiments, suture may not be used to secure the clip body assembly 11 to a portion of the LAA. In those embodiments, the securing element 73 (or securing elements 73) may be any element or combination of elements that secure the first arm body 16 and the second arm body 116 in the engaged position to compress the tissue of the LAA as described. In some embodiments, the securing element 73 (or elements 73) may be a biasing element, such as a spring (not shown), that acts on one or both of the first arm body 16 and the second arm body 116 to secure the first arm body 16 and the second arm body 116 in the engaged position. In other embodiments, one or more adhesives may be applied to one or more surfaces on one or both of the first arm body 16 and the second arm body 116 to secure the first arm body 16 and the second arm body 116 in the engaged position. In other embodiments, one or more lengths of suture may be wrapped or otherwise positioned on the exterior of the first arm body 16 and the second arm body 116, and one or more securing elements 73 may be used to secure any suitable end portion of the sutures to secure the first arm body 16 and the second arm body 116 in the engaged position.

As previously explained, all or a portion of the clip body assembly 11 may be made or manufactured from an absorbable material, such as a bioabsorbable material or a biodegradable material, and any suitable absorbable material or combination of absorbable materials may be used. The absorbable material may also be referred to as a structurally transient material because the structure degrades or dissolves when exposed to the natural healing environment on the beating heart of the patient. In some embodiments, the absorbable material may be an injection moldable material, such as a bioabsorbable polymer. The absorbable material may be any material that allows the clip body assembly 11 to maintain its holding location without migration and compression force losses long enough to enable permanent healing across the compression area of the LAA without leakage during a period of, for example, about one to three months. The time period from the application of the clip body assembly 11 to the time that the compression area of the LAA (or any other atrial wall tissue or cardiac tissue) permanently heals or remodels is known as the healing duration. Further, the absorbable material may be any material that allows the clip body assembly 11 to lose its physical structure by absorption so that it no longer influences the remodeling of the LAA by its physical presence after the permanent healing and exclusion of the distal LAA. For example, the material may be any of the biomaterials previously described or may be Polylactic Acid (PLA), Polyglycolic Acid (PGA), Polycaprolactone (PCL), Polydioxanone (PDS), Polyorthoester, Polyanhydrides, or any combination of these materials. The material may be chosen to target a particular healing duration or to target a specific time or approximate range of time for the structural degradation of the clip body assembly 11 on the LAA.

In some embodiments, all or a portion of the first arm body 16 may be made from a first absorbable material and all or a portion of the second arm body 116 may be made from a second absorbable material. In some embodiments, all or a portion of the hinge portion 60 may be made from the first absorbable material, the second absorbable material, and/or a third absorbable material. In some embodiments, one or both of the first arm body 16 and the second arm body 116 may be made from two or more different absorbable materials, such as a first portion being made from the first absorbable material and a second portion being made from a second absorbable material. In some embodiments, additional portions may be made from additional absorbable materials. The positioning of each portion of the differing absorbable materials may be determined by a particular function of that portion of the first arm body 16 or the second arm body 116, and the differing absorbable materials (along with a correspondingly different cross-sectional shape of the portion, in some embodiments) may achieve a desired pressure profile across the compressed tissue of the LAA during the healing duration.

As previously explained, one or more portions of the first arm body 16 or the second arm body 116 may have a varying cross-sectional shape along the first and second arm body axes 22, 122, respectively, and each of these cross-sectional shapes may be designed to provide sufficient clamping forces across the compressed tissue of the LAA during the healing duration. In some embodiments, these cross-sectional shapes, or portions of these cross-sectional shapes, may be designed to ensure sufficient strength or material durability for the absorbable epicardial clip assembly 10 to remain secured to the LAA while the material degrades. Accordingly, thickness and shapes may be varied across the first and second arm body axes 22, 122, respectively, to result in areas of increased strength, decreased strength, or constant strength across the first and second arm body axes 22, 122. By strengthening one or more specific areas, less absorbable materials may be used to form other areas of the first arm body 16 or the second arm body 116, thereby reducing overall material use and the associated cost.

In some embodiments, a first absorbable material may be used for all or a portion of the engagement surface 32 of the first arm body 16 (and all or a portion of the engagement surface 132 of the second arm body 116), and a second absorbable material (and any further additional absorbable materials) may be used to form the remainder of the first arm body 16 or the second arm body 116, thereby providing differing degradability periods or different structural properties for these different portions of the first arm body 16 or the second arm body 116. In some embodiments, the first absorbable material used for the engagement surfaces 32, 132, may be different than the second absorbable material used on one or more portions of the first arm body 16, the second arm body 116, and/or the hinge 60 that may be engaged or contacted by the previously-discussed introducer device during a procedure. The use of the second absorbable material may be particularly beneficial if the structure of the first arm body 16, the second arm body 116, and/or the hinge 60 is primarily designed for optimal support during the delivery or placement of the clip body assembly 11 on the LAA, but a more rapid relative degradation is desired following the securing of the absorbable epicardial clip assembly 10 to the LAA.

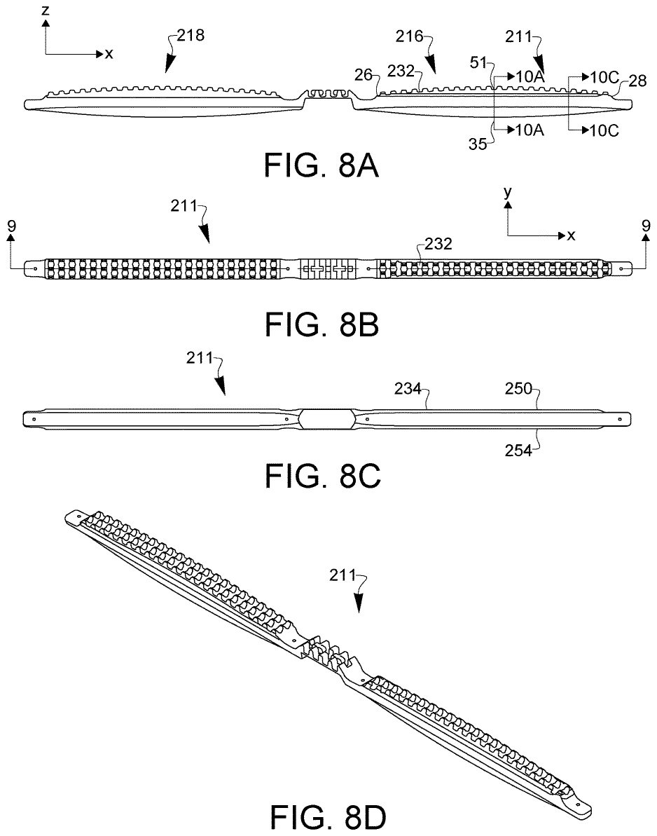

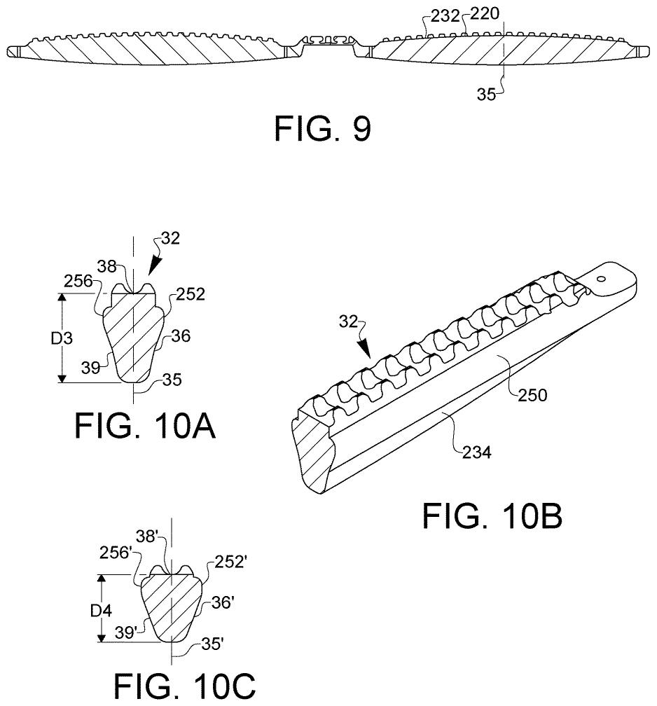

A further embodiment of a clip body assembly 211 is illustrated in FIGS. 8A to 8D. The clip body assembly 211 may be identical to (or substantially identical to) the clip body assembly 11, and like elements will have identical reference numbers. However, the first arm body 216 (and the mirror image, the second arm body 218) of the clip body assembly 211 may have different cross-sectional shapes than the first arm body 16 (and the mirror image, the second arm body 116) of the clip body assembly 11. In particular, the engagement surface 232 of the first arm body 216 may not be planar or substantially planar, but may be cambered or arch-shaped when viewed along an axis parallel to the Y-axis of the reference coordinate system of FIGS. 8A and 8B. That is, when the first arm body 216 is viewed in the cross-sectional view of FIG. 7, which is taken along section line 9-9 of FIG. 8A, an upper edge 220 of the engagement surface 232 may form a cambered, arced, or semi-circular shape from the first end 26 to the second end 28 of the engagement surface 232 (and/or to the first end 18 and second end 20 of the first arm body 216), and this shape may be symmetrical about the axis 35.

Further, the first arm body 216 may have a similar or nearly identical cross-sectional shape (along the X-axis) to the cross-sectional shape of the first arm body 16 of the of clip body assembly 11. However, in some embodiments, some aspects of the cross-sectional shapes may differ. For example, in the cross-sectional views of FIGS. 10A and 10C, which are taken along the section line 10A-10A and 10C-10C of FIG. 8A (and that correspond to the cross-sectional views of FIGS. 4B and 4D previously described), the first arm body 216 may include a first lateral wall upper segment 250 (see FIG. 10B) that may be defined by a first lateral upper edge 252 that may extend downward from a first end portion of an edge 38 of the engagement surface 32 along the Z-axis to the first end of the first lateral edge 36. In addition, the first arm body 216 may include a second lateral wall upper segment 254 (see FIG. 8C) that may be defined by a second lateral upper edge 256 that may extend downward from a second end portion of the edge 38 of the engagement surface 32 along the Z-axis to the first end of the second lateral edge 39.

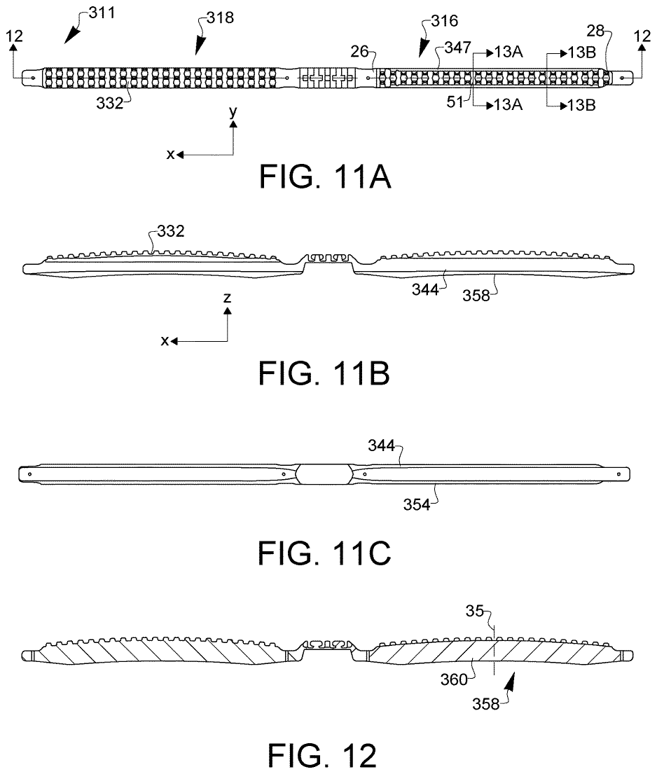

An additional embodiment of a clip body assembly 311 is illustrated in FIGS. 11A to 11C. The clip body assembly 311 may be identical to (or substantially identical to) the clip body assembly 211 (and 11), and like elements will have identical reference numbers. Turning to the clip body assembly 311 in more detail, the engagement surface 332 of the first arm body 316 (and the mirror image, the second arm body 318) of clip body assembly 311 may be cambered or arch-shaped when viewed along an axis parallel to the Y-axis of the reference coordinate system of FIGS. 11A and 11B, which is similar or identical to the engagement surface 232 of the first arm body 216 of the clip body assembly 211 of FIGS. 8A to 8D.

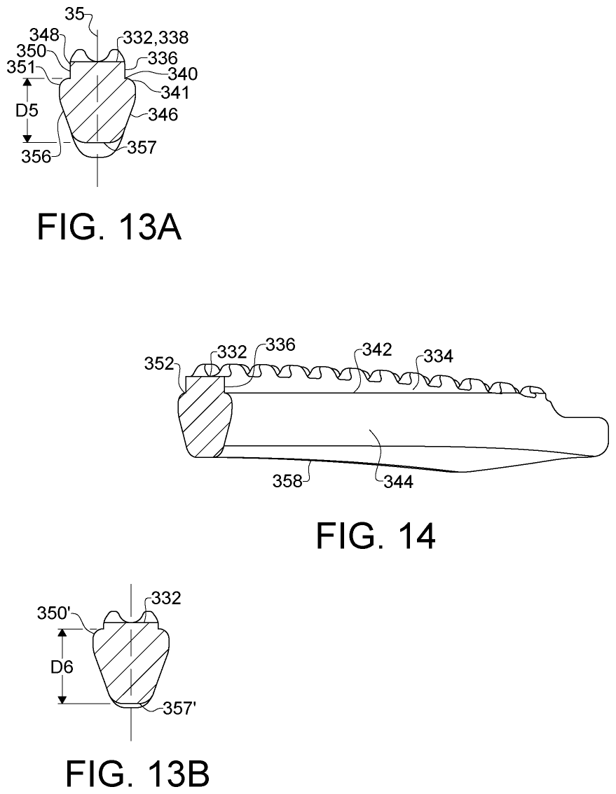

However, the first arm body 316 may have a different cross-sectional shape than the first arm body 216 of the of clip body assembly 211. For example, in the cross-sectional view of FIG. 13A, which is taken along the section line 13A-13A of FIG. 11A that is located at a midpoint 51, the first arm body 316 may include a first lateral wall upper segment 334 (see FIG. 13B) that may be defined by a first lateral upper edge 336 that may extend downward from a first end portion of the edge 338 of the engagement surface 332 along the Z-axis. An end portion of the first lateral upper edge 336 may intersect an edge 340 of a first upper body surface 342 that may be planar or substantially planar, and the first upper body surface 342 may extend from the first end 26 to the second end 28 of the engagement surface 332. In some embodiments, the first upper body surface 342 may intersect the engagement surface 332 at both the first end 26 and the second end 28 of the engagement surface 332. The first arm body 316 may include a first lateral wall 344 (see FIG. 13B) that may be defined by a first lateral edge 346 having a first end that is coupled to an end of the edge 340 of the first upper body surface 342 by a radiused, rounded, or contoured edge portion 341. The first lateral edge 346 may be linear or substantially linear, and may form an acute angle with an axis 35 that is parallel to the Z-axis of the reference coordinate system of FIG. 11B (and that extends through the first body axis 22) such that the first lateral edge 346 converges from the axis 35 as the first lateral edge 346 extends from the first end to the second end. In some embodiments, the first lateral edge 346 may be contoured or arcuate such that the first lateral edge 36 has a shallow concave shape.

Still referring to FIG. 13A, the first arm body 316 may include a second lateral wall upper segment 347 that may be symmetrical to and/or a mirror-image of the first lateral wall upper segment 334 about axis 35. That is, the second lateral wall upper segment 347 may be defined by a second lateral upper edge 348 that may extend downward from a second end portion of the edge 338 of the engagement surface 332 along the Z-axis. An end portion of the second lateral upper edge 348 may intersect an edge 350 of a second upper body surface 352 (illustrated in FIG. 13B) that may be symmetrical to and/or a mirror-image of the first upper body surface 342 about axis 35. That is, the second upper body surface 352 may be planar or substantially planar, and the second upper body surface 352 may extend from the first end 26 to the second end 28 of the engagement surface 332, and in some embodiments, the second upper body surface 352 may intersect the engagement surface 332 at both the first end 26 and the second end 28 of the engagement surface 332. The first arm body 316 may include a second lateral wall 354 (see FIG. 11C) that may be symmetrical to and/or a mirror-image of the first lateral wall 344 about axis 35. That is, the second lateral wall 354 may be defined by a second lateral edge 356 having a first end that is coupled to an end of the edge 350 of the second upper body surface 352 by a radiused, rounded, or contoured edge portion 351. Further, the second lateral edge 356 may be linear or substantially linear, and may form an acute angle with the axis 35 that is parallel to the Z-axis of the reference coordinate system of FIG. 11B (and that extends through the first body axis 22) such that the second lateral edge 356 converges from the axis 35 as the second lateral edge 356 extends from the first end to the second end. In some embodiments, the second lateral edge 356 may be contoured or arcuate such that the first lateral edge 36 has a shallow concave shape.

The first arm body 316 may also include a bottom wall 358 that may be defined by a bottom edge 357 in the cross-sectional view of FIG. 13A. The bottom edge 357 may be planar and the bottom edge may be fifth distance D5 from the second upper body surface 352. First and second contoured edges may couple end points of the bottom edge 357 to the second ends of the first lateral edge 346 and the second lateral edge 356, respectively.

The cross-sectional shape of the first arm body 316 may vary from the midpoint 51 towards the second end 28 of the engagement surface 332. In particular, the distance between the point where the bottom edge 357 intersects an axis (which is parallel to the axis 35 and which extends along the cross-sectional plane) and the edge 350 of the second upper body surface 352 may increase from the midpoint 51 towards the second end 28 of the engagement surface 332 when taken along the first body axis 22. For example, in the cross-sectional view of FIG. 13B, which is taken along the section line 13B-13B of FIG. 11A that is located offset from the second end 28 of the engagement surface 32, a point where the bottom edge 357′ intersects the axis 35′ (that may be parallel to the axis 35 and which extends along the cross-sectional plane indicated by section line 13B-13B of FIG. 11A) may extend a sixth distance D6 from the edge 350′ of the second upper body surface 352′, and this sixth distance D6 may be greater than the fifth distance D5 of FIG. 13A. In some embodiments, the first lateral edge 346′ and the second lateral edge 356′ may each be longer than the first lateral edge 346 and the second lateral edge 356 of FIG. 11A.

The cross-sectional shape of the first arm body 16 may also vary from the midpoint 51 towards the first end 26 of the engagement surface 332. In some embodiments, the cross-sectional shape of the first arm body 316 from the midpoint 51 of the engagement surface 332 to the second end 28 of the engagement surface 332 may be a mirror image of the cross-sectional shape of the first arm body 316 from the midpoint 51 to the first end 26 of the engagement surface 332, and the mirror plane or axis may extend through or along section line 13A-13A of FIG. 11A. So configured, when the first arm body 316 is viewed in the cross-sectional view of FIG. 12, which is taken along section line 12-12 of FIG. 11A, a bottom longitudinal edge 360 of the bottom wall 358 may form a cambered, arced, or semi-circular shape from a first point at or adjacent to the first end 26 of the engagement surface 328 to the a second point at or adjacent to the second end 28 of the engagement surface 332 (such that the bottom wall 358 has a shallow concave shape), and this shape may be symmetrical about the axis 35.

As illustrated in FIG. 11A, the clip body assembly 311 also includes the second arm body 318, and the second arm body 316 may be elongated and may extend from a first end 118 to a second end 120 along a second arm body axis 122. The second arm body 316 may be substantially identical to, but a mirror image of, the first arm body 316 and the structure of the second arm body 318 may include any or all of the structural elements described in terms of the first arm body 316.

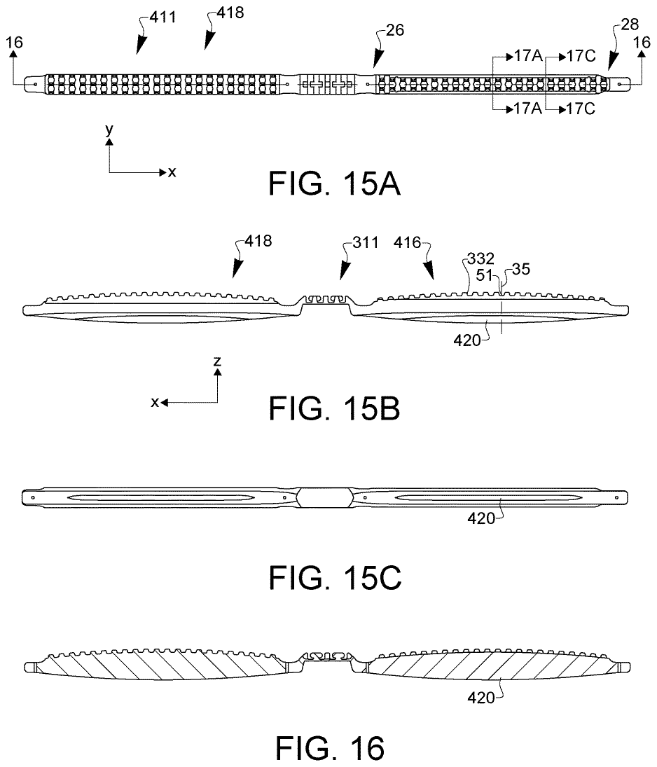

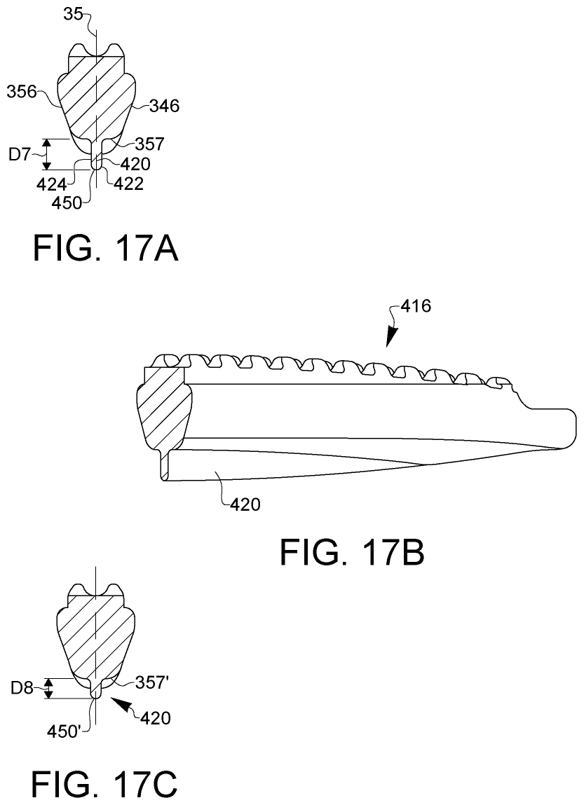

An additional embodiment of a clip body assembly 411 is illustrated in FIGS. 15A to 15C. The clip body assembly 411 may be identical to (or substantially identical to) the clip body assembly 311 and like elements will have identical reference numbers. However, the first arm body 416 may have fin portion 420 that extends from the bottom wall 358 of the first arm body 416. For example, in the cross-sectional view of FIG. 17A, which is taken along the section line 17A-17A of FIG. 15A that is located at a midpoint 51, the fin portion may be defined by a first lateral edge 422 and an opposing second lateral edge 424 that each extend from a corresponding portion of the bottom edge 357. The first lateral edge 422 and an opposing second lateral edge 424 may be parallel, substantially offset, for may converge as the first lateral edge 422 and the second lateral edge 424 extend from the bottom edge 357. The distance between (or the maximum distance between) the first lateral edge 422 and the second lateral edge 424 may be less than a minimum distance between (or a minimum width of) the first lateral edge 346 and the second lateral edge 356. A bottom rounded edge may extend between the first lateral edge 422 and the second lateral edge 424, and a distance between the bottom rounded edge 450 of the fin portion 420 and the bottom edge 357 along axis 35 may be a seventh distance D7.

The cross-sectional shape of the first arm body 316 may vary from the midpoint 51 towards the second end 28 of the engagement surface 332. In particular, the distance between the bottom rounded edge 450 of the fin portion 420 and the bottom edge 357 along an axis parallel to the axis 35 may decrease from the midpoint 51 towards the second end 28 of the engagement surface 332 when taken along the first body axis 22. For example, in the cross-sectional view of FIG. 17C, which is taken along the section line 17C-17C of FIG. 15A that is located offset from the second end 28 of the engagement surface 32, the bottom rounded edge 450′ of the fin portion 420 may extend an eighth distance D8 from the bottom edge 357′ along axis 35′, and the eighth distance D8 may be less than the seventh distance D7.

The cross-sectional shape of the first arm body 416 may also vary from the midpoint 51 towards the first end 26 of the engagement surface 332. In some embodiments, the cross-sectional shape of the first arm body 416 from the midpoint 51 of the engagement surface 332 to the second end 28 of the engagement surface 332 may be a mirror image of the cross-sectional shape of the first arm body 416 from the midpoint 51 to the first end 26 of the engagement surface 332, and the mirror plane or axis may extend through or along section line 17A-17A of FIG. 15A. So configured, when the first arm body 416 is viewed in the cross-sectional view of FIG. 16, which is taken along section line 16 -16 of FIG. 15A, a bottom longitudinal fin edge 460 of the fin portion 420 may form a cambered, arced, or semi-circular shape from a first point at or adjacent to the first end 26 of the engagement surface 328 to the a second point at or adjacent to the second end 28 of the engagement surface 332 (such that the bottom longitudinal fin edge 460 has a shallow convex shape), and this shape may be symmetrical about the axis 35.

As illustrated in FIG. 15A, the clip body assembly 411 also includes the second arm body 418, and the second arm body 416 may be elongated and may extend from a first end 118 to a second end 120 along a second arm body axis 122. The second arm body 416 may be substantially identical to, but a mirror image of, the first arm body 416 and the structure of the second arm body 418 may include any or all of the structural elements described in terms of the first arm body 416.

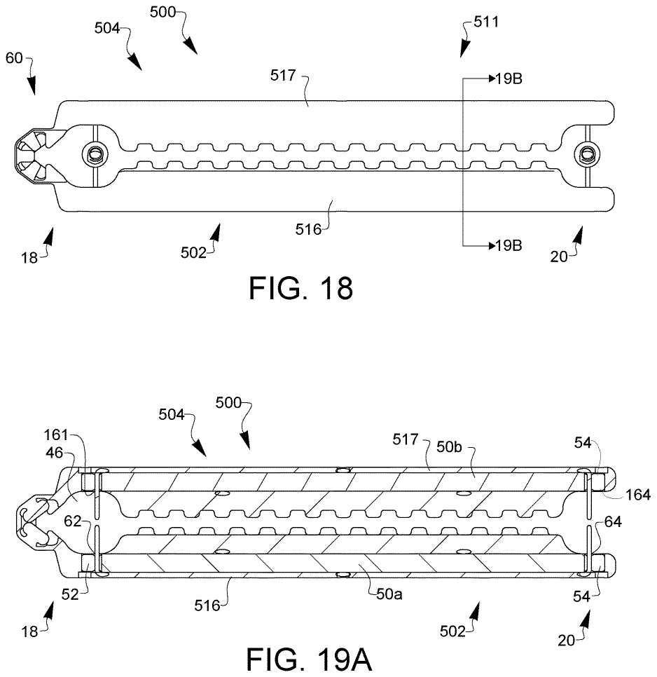

As previously described, one or both of the first arm body 16 and the second arm body 116 may be made from two or more different absorbable materials, such as a first portion being made from the first absorbable material and a second portion being made from a second absorbable material. FIG. 18 illustrates an additional embodiment of the atrial clip assembly 500 may include an embodiment of a clip body assembly 511 comprising two or more different absorbable materials. The clip body assembly 511(and the atrial clip assembly 500) may be similar (or substantially similar) to the clip body assembly 211, 311 (and the atrial clip assembly 10) and like elements will have identical reference numbers.

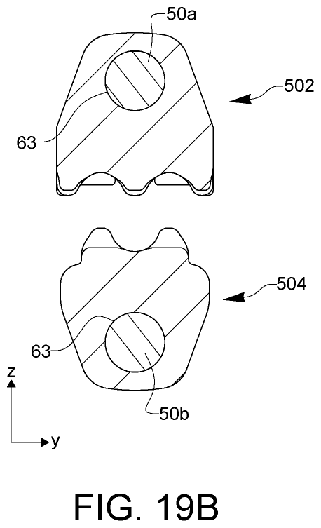

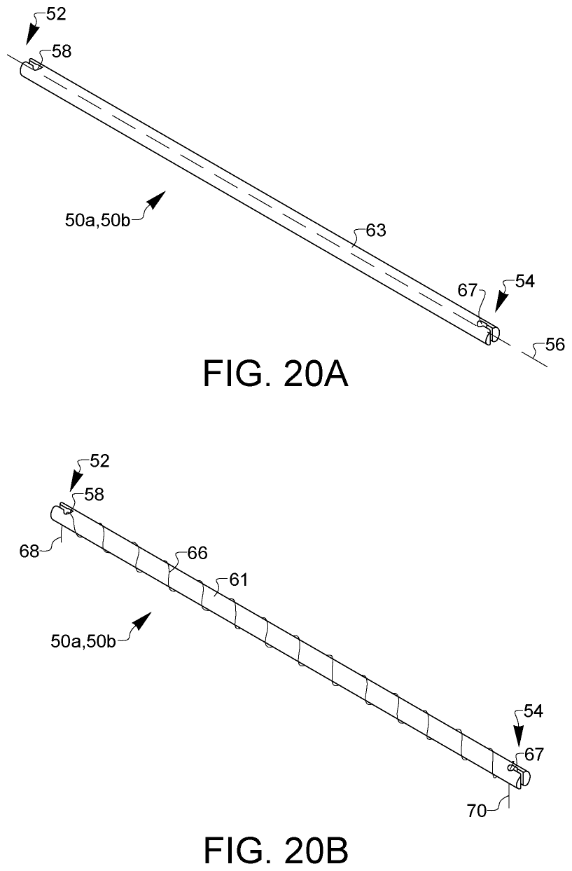

In particular, the clip body assembly 511 may include the first arm assembly 502 which may include the first arm body 516 which may extend from the first end 18 to the second end 20 along the first arm body axis 22 (see FIG. 2A). As illustrated in the cross-sectional view of FIG. 19A, which is taken along a section line equivalent to section line 4A-4A of FIG. 2A, the first arm assembly 502 may also include a first spine portion 50a that extends along all or a portion of the first arm body 516. As illustrated in the isolated perspective view of the first spine portion 50a of FIG. 20A, the first spine portion 50a may be elongated and may extend from a first end 52 to a second end 54 along a first spine axis 56 that may be aligned with the first arm body axis 22. The first end 52 may be at or adjacent to (e.g., slightly offset from) the first end 18 of the first arm body 516, and the second end 54 may be at or adjacent to (e.g., slightly offset from) the second end 20 of the first arm body 516. The first spine portion 50a may have any suitable cross-sectional shape or combination of shapes. For example, as illustrated in FIG. 20A and the cross-sectional view of FIG. 19B, the first spine portion 50a may have an exterior surface 63 having a cylindrical shape which may be uniform or substantially uniform from the first end 52 (or from a point adjacent to the first end 52) to the second end 54 (or to a point adjacent to the second end 54). The first spine portion 50a may have a first aperture 58 adjacent to the first end 52, and the first aperture 58 may extend along an axis that is normal to the first spine axis 56. The first spine portion 50a may also have a second aperture 67 adjacent to the second end 54, and the second aperture 67 may extend along an axis that is normal to the first spine axis 56.

In some embodiments, the first arm body 516 may be made from or comprise a first absorbable material, and the first spine portion 50a may be made from or comprise a second absorbable material that is different than the first absorbable material. For example, the first spine portion 50a may be fabricated or made from or of an absorbable material that is stronger, stiffer, or more rigid (or, alternatively, less rigid) than the absorbable material of the first arm body 516. Alternatively, or additionally, the first spine portion 50a may be fabricated or made from or of an absorbable material that has a faster (or slower) degradation rate than the absorbable material of the first arm body 516. In some embodiments, the first spine portion 50a may be made from or comprise polyglycolic acid (PGA) and the first arm body 516 may be made from or comprise polydioxanone (PDO). The first spine portion 50a or the first arm body 516 may also be Poly-L-lactic acid (PLLA), polyethylene glycol (PEG), polycaprolactone (PCL). In some embodiments, all or a portion of the first arm body 516 may be coated with a coating that may affect the rate of absorption. For example, all or a portion of the first arm body 516 (and the hinge portion 60 and the second arm body 517) may be coated with a hydrophobic coating or a hydrophilic coating.

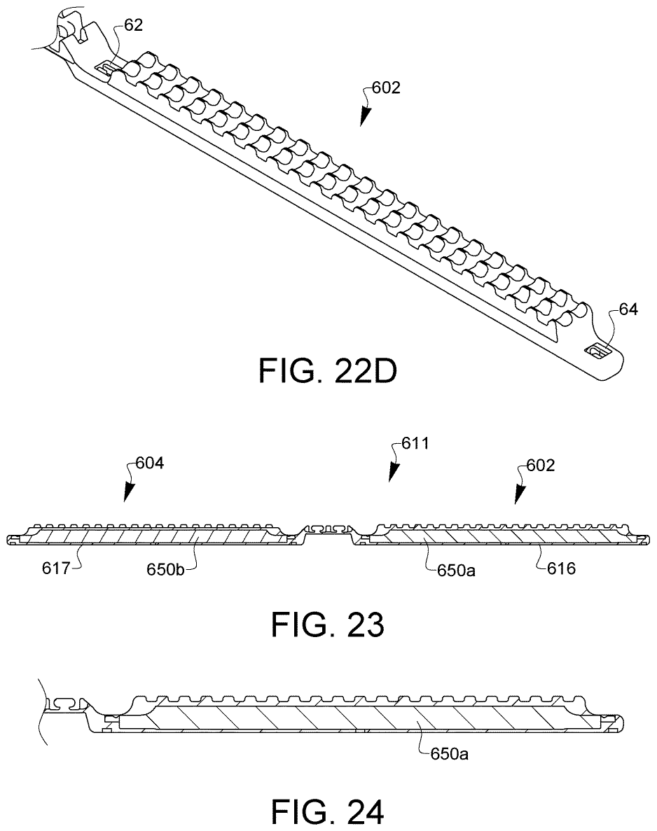

The first spine portion 50a may be incorporated into or coupled to the first arm body 516 in any suitable manner, such as by co-molding the first spine portion 50a with (or within a portion of) the first arm body 516. For example, the first spine portion 50a may be placed in a mold used to form the first arm body 516, and the part of the first arm body 516 may be formed around to surround the first spine portion 50a. As such, a first arm aperture 62 (see FIG. 22D) may be formed on a surface at least partially defining the first notch edge portion 46 of the first arm body 516 (e.g., at or adjacent to the first end 18) and the first arm aperture 62 may be aligned with the first aperture 58 of the first spine portion 50a (see FIG. 20A). Further, a second arm aperture 64 (see FIG. 22D) may be formed on a surface at least partially defining the second notch edge portion 48 of the first arm body 516 (e.g., at or adjacent to the second end 20) and the second arm aperture 64 may be aligned with second aperture 67 (see FIG. 20A) of the first spine portion 50a.

In some embodiments of the atrial clip assembly 500, the first arm assembly 502 may also include one or more portions of suture 66 may be wrapped around the exterior surface 63 of the first spine portion 50a, as illustrated in FIG. 20B, prior to molding the first arm body 516 over the first spine portion 50a such that the one or more portions of suture 66 disposed within the first arm body 516 are prevented from displacing relative to the first arm body 516 and are anchored by the first arm body 516. The one or more portions of suture 66 may be wrapped around the exterior surface 63 of the first spine portion 50a in any suitable manner, and the one or more portions of suture 66 may be wrapped around the exterior surface 63 of the first spine portion 50a in a helical manner, for example, as illustrated in FIG. 20B. After molding, the first end 68 of the one or more portions of suture 66 may extend through the first arm aperture 62 of the first arm body 516 and all or a portion of the first aperture 58 of the first spine portion 50a. Also after molding, the second end 70 of the one or more portions of suture 66 may extend through the second arm aperture 64 of the first arm body 516 and all or a portion of the second aperture 67 of the first spine portion 50a. The one or more portions of suture 66 may be any material or combination of materials capable of securing (or cooperating to secure) the first arm assembly 502 to the second arm assembly 504, as previously described. As such, the one or more portions of suture 66 may be any suture, wire, thread, fabric, or cable, for example.

As illustrated in the FIG. 18, the clip body assembly 511 may also include the second arm assembly 504 may also include a second spine portion 50b that extends along all or a portion of the second arm body 517. The second spine portion 50b, may be identical to the first spine portion (illustrated in FIG. 20A) and the first end 52 of the second spine portion 50b may be at or adjacent to (e.g., slightly offset from) the first end 126 of the second arm body 517, and the second end 54 of the second spine portion 50b may be at or adjacent to (e.g., slightly offset from) the second end 128 of the second arm body 517. The material of the second arm body 517 and/or the second spine portion 50b may be identical to the material of the first arm body 516 and/or the first spine portion 50a, and all or a portion of the second arm body 517 may be coated with the same coating as the first arm body 516.

Similarly or identically to the first spine portion 50a, the second spine portion 50b may be co-molded with the second arm body 517 in any suitable manner. For example, the second spine portion 50b may be placed in a mold used to form the second arm body 517, and the part of the second arm body 517 may be formed around to surround the second spine portion 50b. As such, with reference to FIG. 19A, a first arm aperture 161 may be formed on a surface at least partially defining the first notch edge portion 146 of the second arm body 517 (e.g., at or adjacent to the first end 126 of the second arm body 517) and the first arm aperture 161 may be aligned with the first aperture 58 of the second spine portion 50b. Further, a second arm aperture 164 may be formed on a surface at least partially defining the second notch edge portion 148 of the second support portion 124 (e.g., at or adjacent to the second end 128 of the second arm body 517) and the second arm aperture 164 may be aligned with second aperture 67 of the second spine portion 50b.

In some embodiments of the atrial clip assembly 500, the second arm assembly 504 may also include one or more portions of suture 166 that may be wrapped around an exterior surface 63 of the second spine portion 50b prior to molding the second arm body 517 over the second spine portion 50b, and the one or more portions of suture 166 wrapped around the exterior surface 63 of the second spine portion 50b may be identically configured to the one or more portions of suture 66 that may be wrapped around an exterior surface 63 of the first spine portion 50a previously discussed and as illustrated in FIG. 20B. After molding, a first end 168 of the one or more portions of suture 166 may extend through the first arm aperture 161 of the second arm body 517 and all or a portion of the first aperture 58 of the second spine portion 50b. Also after molding, a second end 170 of the one or more portions of suture 166 may extend through the second arm aperture 164 of the second arm body 517 and all or a portion of the second aperture 67 of the second spine portion 50b.

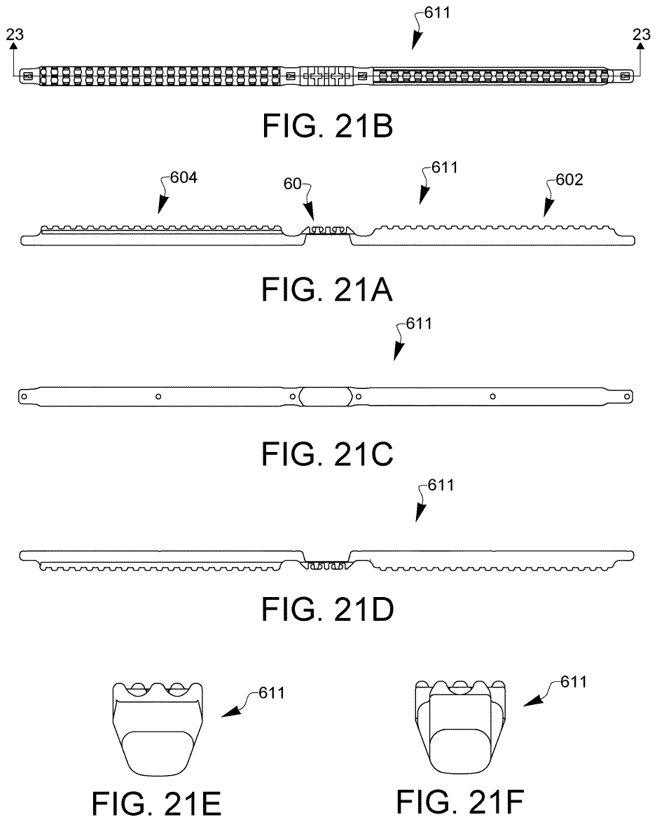

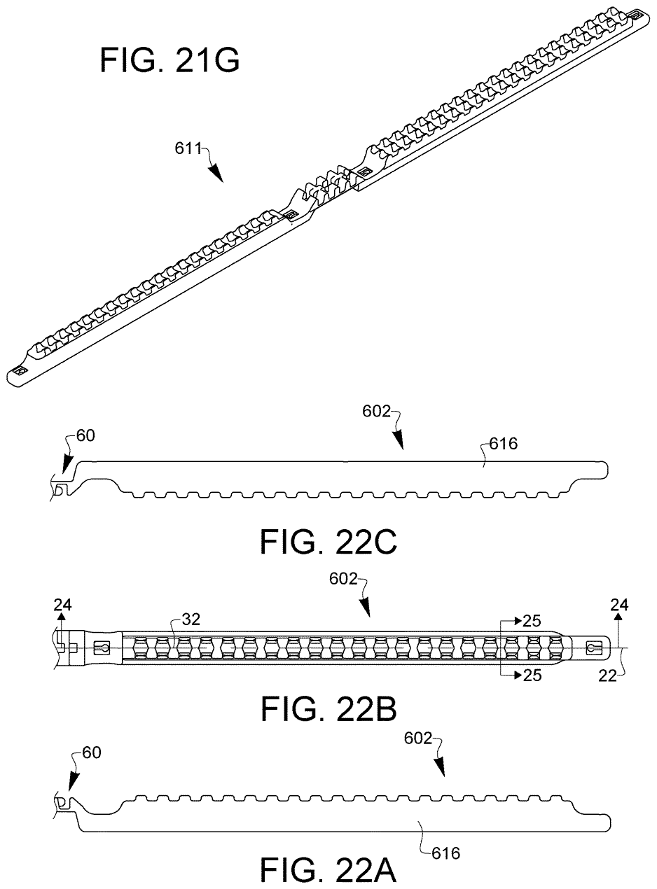

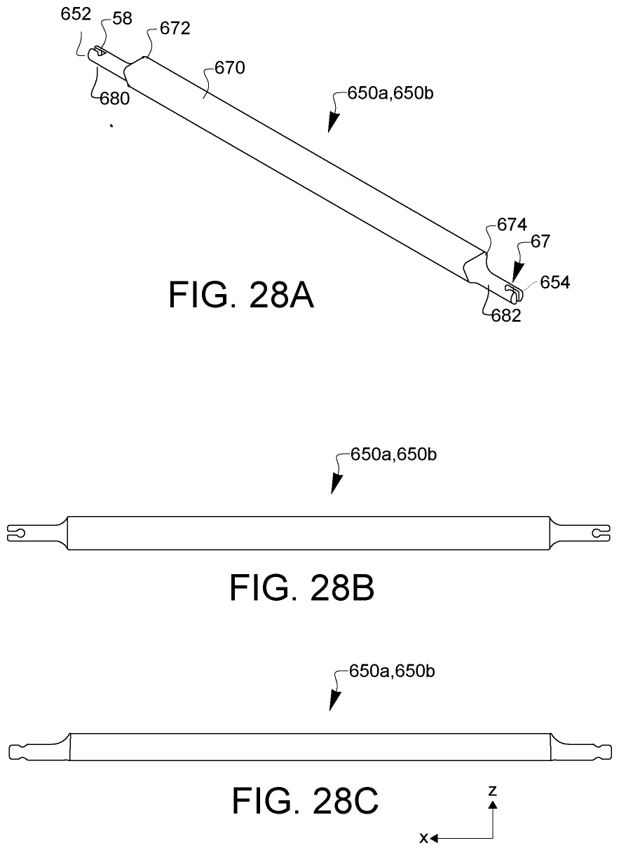

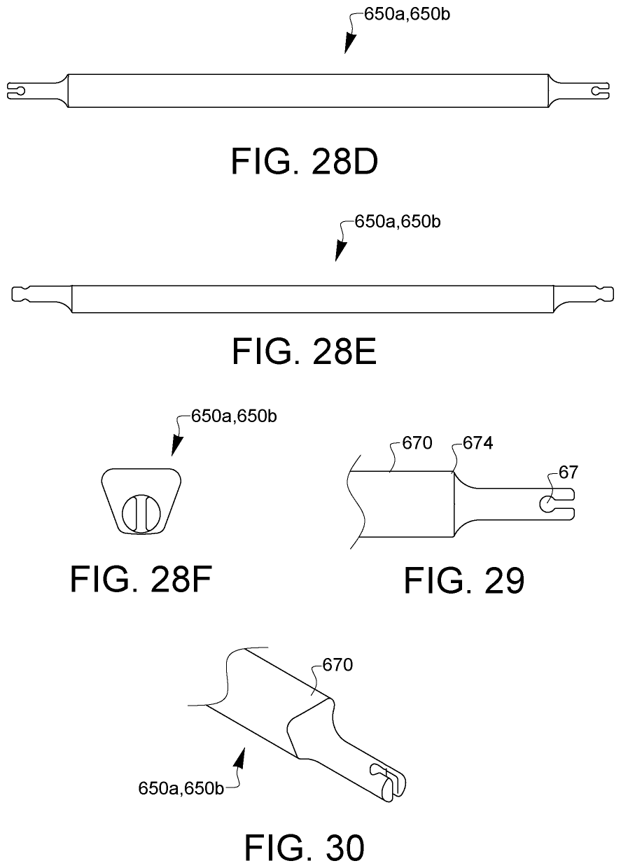

An additional embodiment of a clip body assembly 611 is illustrated in FIGS. 21A to 21G. The clip body assembly 611 may be identical to (or substantially identical to) the clip body assembly 211, 311 or 511 and like elements will have identical reference numbers. However, the clip body assembly 611 may have a first spine portion 650a and a second spine portion 650b that may have a non-circular cross-sectional shape. In particular, as illustrated in the various views of FIGS. 28A to 28F, the first spine portion 650a may be elongated and may extend from a first end 652 to a second end 654 along a first spine axis (not shown, but identical to the first spine axis 56 of FIG. 20A), and the first spine axis may extend parallel to the X-axis of the reference coordinate system of FIG. 28C. With reference to FIG. 28A, the first spine portion 650a may also include a spine body 670 that may extend from a first end 672 to a second end 674 along the first spine axis. A first end projection 680 may extend from the first end 672 of the spine body 670 to the first end 652 of the first spine portion 650a, and a second end projection 682 may extend from the second end 674 of the spine body 670 to the second end 654 of the first spine portion 650a. The first end projection 680 may include the first aperture 58 adjacent to the first end 652 of the first spine portion 650a, and the second end projection 680 may include, and the second aperture 67 adjacent to the second end 654 of the first spine portion 650a.

After molding, the first end 68 of the one or more portions of suture 66 may extend through the first arm aperture 62 of the first arm body 516 and all or a portion of the first aperture 58 of the first spine portion 50a, similar to the configuration of FIG. 19. Also after molding, the second end 70 of the one or more portions of suture 66 may extend through the second arm aperture 64 of the first arm body 516 and all or a portion of the second aperture 67 of the first spine portion 50a.

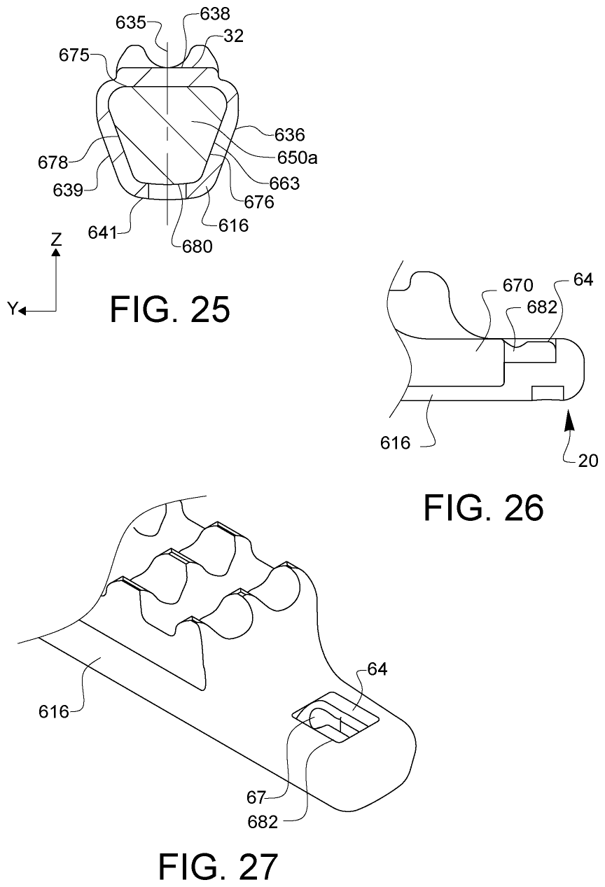

The spine body 670 may have any suitable cross-sectional shape (normal to first spine axis) or combination of cross-sectional shapes from the first end 672 to the second end 674. For example, as illustrated in the cross-sectional shape of the first arm assembly 602 illustrated in FIG. 25, the cross-sectional shape of the spine body 670 may generally conform to the cross-sectional shape of the first arm body 616, and the cross-sectional shape of the spine body 670 may generally conform to the cross-sectional shape of the first arm body 616 as the cross-sectional shape of the first arm body 616 may change along the length of the first arm body 616. However, the cross-sectional shape of the spine body 670 may be constant from the first end 672 to the second end 674.

In some embodiments, such as the embodiment of FIG. 25, the cross-sectional shape of the spine body 670 may may be partially defined by a top edge 675 that may be parallel to the Y-axis of the reference coordinate system of FIG. 25. A first lateral edge 676 may extend from a first end to a second end, and the first end may be coupled to a first end of the top edge 675 by a rounded or contoured segment. The first lateral edge 676 may be linear or substantially linear, and may form an acute angle with an axis 635 that is parallel to the Z-axis of the reference coordinate system of FIG. 25 such that the first lateral edge 676 diverges from the axis 635 as the first lateral edge 676 extends from the second end towards the first end.

Still referring to FIG. 25, the cross-sectional shape of the spine body 670 may may be further defined by a second lateral edge 678 in the cross-sectional view. The second lateral edge 678 may have any suitable shape or combination of shapes and may differ in shape from the first lateral edge 676. However, the second lateral edge 678 may be a mirror image of the first lateral edge 676 taken about the axis 635. That is, the second lateral edge 678 may extend from a first end to a second end, and the first end may be coupled to a second end of the top edge 675 by a rounded or contoured segment. The second lateral edge 678 may be linear or substantially linear, and may form the acute angle (or any other acute angle) with the axis 635 such that the second lateral edge 678 diverges from the axis 635 as the second lateral edge 678 extends from the second end towards the first end.

The cross-sectional shape of the spine body 670 may may be additionally defined by a bottom edge 680, and the bottom edge 680 may extend from the second end of the first lateral edge 766 to the second end of the second lateral edge 678, and the bottom edge 680 may be symmetrical about the axis 635. In some embodiments, the bottom edge 680 may have a semicircular or arcuate shape. So configured, and as illustrated in the cross-sectional view of FIG. 25, the top edge 675, first lateral edge 676, second lateral edge 678, and bottom edge 680 may be offset an equal (or substantially equal) distance form a corresponding top edge 638, first lateral edge 636, second lateral edge 639, and bottom edge 641, respectively, of the cross-sectional shape of first arm body 616.

Various advantages of the epicardial clip assembly have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

1. An absorbable epicardial clip assembly for occluding a left atrial appendage, the absorbable epicardial clip assembly comprising:
a clip body assembly comprising:
an elongated first arm body extending from a first end to a second end along a first arm body axis, the first arm body including a first engagement surface;
an elongated second arm body extending from a first end to a second end along a second arm body axis, the second arm body including a second engagement surface; and
a hinge portion having a first end coupled to the first end of the first arm body and a second end coupled to the first end of the second arm body such that the first arm body and the second arm body are configured to pivot about the hinge portion from a disengaged position, in which a first portion of the left atrial appendage may be positioned between the first engagement surface and the second engagement surface, to an engaged position, in which the second end of the first arm body is secured to the second end of the second arm body and in which the first engagement surface and the second engagement surface are configured to remain engaged with and sufficiently compress opposing tissue surfaces of the first portion of the left atrial appendage to induce ischemic necrosis along the portion of the left atrial appendage and to cut off the blood flow distal to the portion of the left atrial appendage, thereby leading to left atrial appendage tissue death and reabsorption,
wherein the first arm body, the second arm body, and the hinge portion comprising a first absorbable material such that the clip body assembly is configured to degrade within a first period of time after being secured in the engaged position, thereby leaving little or no mechanical residuals to impede long-term heart remodeling and such that the clip body assembly maintains structural integrity to provide occlusion and ischemia during the first period of time.

2. The absorbable epicardial clip assembly of claim 1, wherein the first period of time is not less than two weeks and not more than two years.

3. The absorbable epicardial clip assembly of claim 1, wherein a first cross-sectional shape of the first arm body at a first point along the first arm body axis is different than a second cross-sectional shape of the first arm body at a second point along the first arm body axis such that a desired pressure profile results across the first engagement surface when the first arm body and the second arm body are in the engaged position.

4. The absorbable epicardial clip assembly of claim 3, wherein a third cross-sectional shape of the first arm body at a third point along the first arm body axis is different than each of the first cross-sectional shape and the second cross-sectional shape.

5. The absorbable epicardial clip assembly of claim 1, further comprising:
a first length of suture having a first portion that extends from a first end to a second end within a first portion of the first arm body and a first end portion coupled to the first end of the first portion, the first end portion extending from a second portion of the first arm body; and
a second length of suture having a first portion that extends from a first end to a second end within a first portion of the second arm body and a first end portion coupled to the first end of the first portion, the first end portion extending from a second portion of the second arm body, wherein the first end portion of the first length of suture is configured to be coupled to the first end portion of the second length of suture to secure the clip body assembly in the engaged position.

6. The absorbable epicardial clip assembly of claim 5, further comprising a fastener that is configured to couple the first end portion of the first length of suture and the first end portion of the second length of suture, and wherein the fastener is made from a second absorbable material.

7. The absorbable epicardial clip assembly of claim 6, wherein the first length of suture and the second length of suture are each made or formed from a third absorbable material.

8. The absorbable epicardial clip assembly of claim 5, wherein the first portion of the first length of suture is insert molded within the first portion of the first arm body, and the first portion of the second length of suture is insert molded within the first portion of the second arm body.

9. The absorbable epicardial clip assembly of claim 1, the clip body assembly further comprising:
a first spine portion extending from a first end to a second end along a first spine axis, and wherein the first spine portion extends within a corresponding portion of the first arm body; and
a second spine portion extending from a first end to a second end along a second spine axis, wherein the second spine portion extends within a corresponding portion of the second arm body,
wherein the first spine portion and the second spine portion comprising a second absorbable material such that the first spine portion and the second spine portion are configured to degrade within a second period of time after being secured in the engaged position.

10. The absorbable epicardial clip assembly of claim 9, further comprising:
a first suture, wherein a first portion of the first suture is disposed around a portion of the first spine portion, and wherein a second portion of the first suture extends external to the first arm body; and
a second suture, wherein a first portion of the second suture is disposed around a portion of the second spine portion, and wherein a second portion of the second suture extends external to the second arm body, and wherein the second portion of the first suture is configured to be coupled to the second portion of the second suture to secure the absorbable epicardial clip assembly in the engaged position.

11. The absorbable epicardial clip assembly of claim 1, wherein in the engaged position of the first arm body and the second arm body, the first arm body axis is parallel to the second arm body axis.

12. A method of occluding a left atrial appendage with an absorbable epicardial clip assembly, the method comprising:
positioning a clip body assembly of the absorbable epicardial clip assembly along a portion of the left atrial appendage, wherein the clip body assembly includes an elongated first arm body that is coupled to an elongated second arm body by a hinge portion, and wherein the portion of the left atrial appendage is disposed between a first portion of the first arm body and a first portion of the second arm body; and
securing a first end of the first arm body to a first end of the second arm body such that the first portion of the first arm body and the first portion of the second arm body remain engaged with and sufficiently compress the tissue of the portion of the left atrial appendage to induce ischemic necrosis along the portion of the left atrial appendage and to cut off the blood flow distal to the portion of the left atrial appendage, thereby leading to left atrial appendage tissue death and reabsorption,
wherein the clip body assembly is configured to degrade within a first period of time after securing the first end of the first arm body to the first end of the second arm body, thereby leaving little or no mechanical residuals to impede long-term heart remodeling and such that the clip body assembly maintains structural integrity to provide occlusion and ischemia during the first period of time.

13. The method of occluding the left atrial appendage with the absorbable epicardial clip assembly of claim 12, wherein the first period of time is not less than two weeks and not more than two years.

14. The method of occluding the left atrial appendage with the absorbable epicardial clip assembly of claim 12, wherein the absorbable epicardial clip assembly includes a first portion of suture coupled to the first body arm and a second potion of suture coupled to the second body arm, and
wherein securing the first end of the first arm body to the first end of the second arm body includes securing an end of the first portion of suture to an end of the second portion of suture.

15. The method of occluding the left atrial appendage with the absorbable epicardial clip assembly of claim 14, wherein the absorbable epicardial clip assembly further comprises a suture fastener, and
wherein securing the end of the first portion of suture to the end of the second portion of suture includes securing the end of the first portion of suture to the end of the second portion of suture by the fastener.

16-50. (canceled)
